(19) United States
(12) Patent Application Publication
(10) Pub. No.: US 2006/0224110 A1
(43) Pub. Date: Oct. 5, 2006

(54) Methods for minimally invasive vascular access

(76) Inventors: Michael J. Scott, Lake Forest, CA (US); Branislav Radovancevic, Houston, TX (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/083,042

(22) Filed: Mar. 17, 2005

(51) Int. Cl. A61M 31/00; A61M 37/00

(52) U.S. Cl. 604/95.01; 604/506

(57) ABSTRACT

The present methods provide access to high flow vessels without causing severe trauma for the patient. At the same time, the methods maximize the size of a vascular instrument that can be deployed at the target location. The methods involve tunneling through the patient's tissue to create an access path between a percutaneous access site and the target location by using a second percutaneous site that is generally subcutaneous.

### BACKGROUND OF THE INVENTION

1. Field of the Invention

The present invention relates generally to a method of providing extracorporeal access to a patient's vasculature and, more specifically, to accessing a high volume main vessel by first accessing a low volume peripheral vessel.

2. Description of the Related Art

When inserting a cannula into a patient, it may be advantageous to access a vessel having a high volumetric flow capacity. For example, when applying the cardiac assist system described in U.S. Pat. No. 6,685,621, it may be advantageous to locate the inflow and outflow cannulae in one or more high flow vessels. The larger the vessel, the larger the vascular instrument that may be deployed there. Unfortunately, certain high flow vessels, such as those located in the abdominal cavity, are buried deep beneath bodily tissue and organs. Therefore, these vessels are often difficult to access. The difficulty in accessing these vessels is exacerbated by the fact that it is difficult to precisely identify the location of such deeply buried vessels from outside the patient's body.

Traditionally, these vessels have been accessible through a surgical cut down. Such a procedure is highly invasive and traumatic for the patient, requiring a lengthy recovery period including hospitalization. Alternatively, high flow vessels have been accessible through peripheral vessels having lower volumetric flow capacities. For example, to access the descending aorta, a physician may insert a cannula percutaneously into the patient's femoral artery, then advance the cannula upstream into the aorta. The femoral artery is advantageously located subcutaneously near the patient's skin surface, and is easily accessible without the need for a traumatic surgical cut down. Unfortunately, the relatively low volumetric flow capacity of the femoral artery limits the size of the cannula that can be deployed through that access location.

### SUMMARY OF THE INVENTION

Accordingly, a method of accessing a high flow vessel without causing severe trauma to the patient, while maximizing the size of a cannula to be deployed in the vessel, would be of great benefit to patients undergoing vascular procedures.

The preferred embodiments of the present methods for minimally invasive vascular access have several features, no single one of which is solely responsible for their desirable attributes. Without limiting the scope of these methods as expressed by the claims that follow, their more prominent features will now be discussed briefly. After considering this discussion, and particularly after reading the section entitled “Detailed Description of the Preferred Embodiments,” one will understand how the features of the preferred embodiments provide advantages, which include the capability to access high flow vessels without causing severe trauma to the patient, while maximizing the size of a vascular instrument to be deployed in the vessel.

A preferred method of the present invention provides minimally invasive access to a deeply buried target location in a patient's vasculature. Because the target location is buried deep beneath the patient's skin, a relatively large amount of bodily tissue and/or organs lies between a first percutaneous site and the target location. The present inventive method permits relatively easy access to the target location from a second percutaneous site where the vasculature is located relatively close to the skin. The vasculature at the target location includes a vessel segment with a first perimeter that is larger than a second perimeter of a second vessel segment located near the second percutaneous site. The volumetric flow rate at the first vessel segment is significantly higher than at the second vessel segment in some applications. The method comprises the steps of puncturing a patient's skin and vasculature with a needle at the second percutaneous site, inserting a guide wire through the needle and into the vasculature at the second percutaneous site, removing the needle from the vasculature, advancing the guide wire through the vasculature, with the aid of a visualization apparatus, to the target location, advancing a dilator over the guide wire and inserting the dilator into the vasculature at the second percutaneous site, thereby widening an opening in the vasculature at the second percutaneous site, advancing a tunneling device having a cover through the vasculature, with the further aid of the visualization apparatus, along the guide wire from the second percutaneous site to the target location, the tunneling device being configured to be steerable and having a distal point capable of penetrating the vasculature and tissue between the vasculature and the skin, the cover protecting the vasculature as the device travels through the vasculature, piercing the vasculature wall with the tunneling device at the target location and advancing the tunneling device through the vasculature wall and through the patient's tissue, with the further aid of the visualization apparatus, avoiding sensitive bodily structures, to the first percutaneous site, and inserting a cannula through the first percutaneous site, through the patient's tissue, and into the vasculature at the target location.

An alternative method comprises the steps of puncturing a patient's skin with a needle at a percutaneous site above a deeply buried target region of the vasculature, inserting a tunneling device through the patient's skin at the percutaneous site, advancing the tunneling device through the patient's tissue beneath the percutaneous insertion site, with the aid of a visualization apparatus, so as to avoid sensitive bodily structures, to the vasculature, thereby creating a pathway from the percutaneous insertion site to the vasculature proximate the target location, removing the tunneling device from the pathway, advancing a sheath, with the further aid of the visualization apparatus, along the pathway to the vasculature proximate the target location, an end of the sheath including apparatus configured to capture the vasculature, capturing the vasculature with the capturing apparatus and with the further aid of the visualization apparatus, advancing a guide wire through the sheath to the vasculature, with the further aid of the visualization apparatus, advancing a needle along the guide wire to the vasculature, with the further aid of the visualization apparatus, piercing a wall of the vasculature with the needle, and with the further aid of the visualization apparatus, to produce a vascular opening, advancing the guide wire through the vascular opening, with the further aid of the visualization apparatus, and into the vasculature at the target location, removing the needle from the vascular opening, advancing a dilator along the guide wire, with the further aid of the visualization apparatus, and through the vascular opening to widen the opening, advancing a cannula along the guide wire, with the further aid of the visualization apparatus, through the vascular opening, and into the vasculature at the target location.

Various apparatuses described and claimed below can be adapted for one or more aspects of the foregoing methods and variations thereof, including the variations discussed and claimed below. In one embodiment, a tunneling device is provided that permits a clinician to reach a target location of a patient's vasculature from a percutaneous site that is remote from the target location. The target location usually is deeper than subcutaneous. The tunneling device includes an elongate portion configured to pass through the patient's vasculature. The elongate portion has a distal end configured to cut through the vasculature and tissue.

### BRIEF DESCRIPTION OF THE DRAWINGS

FIG. 1 is a schematic view of one embodiment of a heart assist system having multiple conduits for multi-site application, shown applied to a patient's vascular system;

FIG. 2 is a schematic view of another application of the embodiment of FIG. 1;

FIG. 3 is a schematic view of another embodiment of a heart assist system having multiple conduits for multi-site application wherein each of the conduits is applied to more than one vessel, shown applied to a patient's vascular system;

FIG. 4 is a schematic view of another embodiment of a heart assist system having multiple conduits for multi-site application and employing a connector with a T-shaped fitting, shown applied to a patient's vascular system;

FIG. 5 is a schematic view of an L-shaped connector coupled with an inflow conduit, shown inserted within a blood vessel;

FIG. 6 is a schematic view of another embodiment of a heart assist system having multiple conduits for multi-site application, shown applied to a patient's vascular system;

FIG. 7 is a schematic view of another application of the embodiment of FIG. 6, shown applied to a patient's vascular system;

FIG. 8 is a schematic view of another application of the embodiment of FIG. 6, shown applied to a patient's vascular system;

FIG. 9 is a schematic view of another embodiment of a heart assist system having multiple conduits for multi-site application, a reservoir, and a portable housing for carrying a portion of the system directly on the patient;

FIG. 10 is a schematic view of another embodiment of a heart assist system having a multilumen cannula for single-site application, shown applied to a patient's vascular system;

FIG. 11 is a schematic view of a modified embodiment of the heart assist system of FIG. 10, shown applied to a patient's vascular system;

FIG. 12 is a schematic view of another embodiment of a heart assist system having multiple conduits for single-site application, shown applied to a patient's circulatory system;

FIG. 13 is a schematic view of another application of the embodiment of FIG. 12, shown applied to a patient's vascular system;

FIG. 14 is a schematic view of one application of an embodiment of a heart assist system having an intravascular pump enclosed in a protective housing, wherein the intravascular pump is inserted into the patient's vasculature through a non-primary vessel;

FIG. 15 is a schematic view of another embodiment of a heart assist system having an intravascular pump housed within a conduit having an inlet and an outlet, wherein the intravascular pump is inserted into the patient's vasculature through a non-primary vessel; and

FIG. 16 is a schematic view of a modified embodiment of the heart assist system of FIG. 15 in which an additional conduit is shown adjacent the conduit housing the pump, and in which the pump comprises a shaft-mounted helical thread.

FIGS. 17A-D are schematic views of various tunneling device for use with the present inventive methods of vasculature access.

FIGS. 18A-F are schematic views of tunneling apparatus tips.

FIGS. 19A-B are schematic views of one embodiment of a tunneling device with an extendable cutter.

FIG. 20 is a schematic view of another embodiment of a tunneling device with an extendable cutter.

FIG. 21A-B are schematic views of a steerable tunneling device.

FIGS. 22A-22B are embodiments of electrode tunneling devices.

### DETAILED DESCRIPTION OF THE PREFERRED EMBODIMENTS

Turning now to the drawings provided herein, more detailed descriptions of various embodiments of heart assist systems and cannulae for use therewith are provided below. Following this discussion, preferred embodiments of the present methods for minimally invasive vascular access are described, along with various apparatuses that can be used to practice the methods.

I. Heart Assist Systems and Cannulae for Use Therewith

Below, a variety of cannulae and cannula assemblies are described that can be used in connection with a variety of heart assist systems that supplement perfusion. Such systems preferably are extracardiac in nature. In other words, the systems supplement blood perfusion, without the need to interface directly with the heart and aorta. Thus, the systems can be applied without major invasive surgery. The systems also lessen the hemodynamic burden or workload on the heart by reducing afterload, impedence, and/or left ventricular end diastolic pressure and volume (preload). The systems also advantageously increase peripheral organ perfusion and provide improvement in neurohormonal status. As discussed more fully below, the systems can be applied using one or more cannulae, one or more vascular grafts, and a combination of one or more cannulae and one or more vascular grafts. For systems employing cannula(e), the cannula(e) can be applied through multiple percutaneous insertion sites (sometimes referred to herein as a multi-site application) or through a single percutaneous insertion site (sometimes referred to herein as a single-site application).

A. Heart Assist Systems and Methods Employing Multi-Site Application

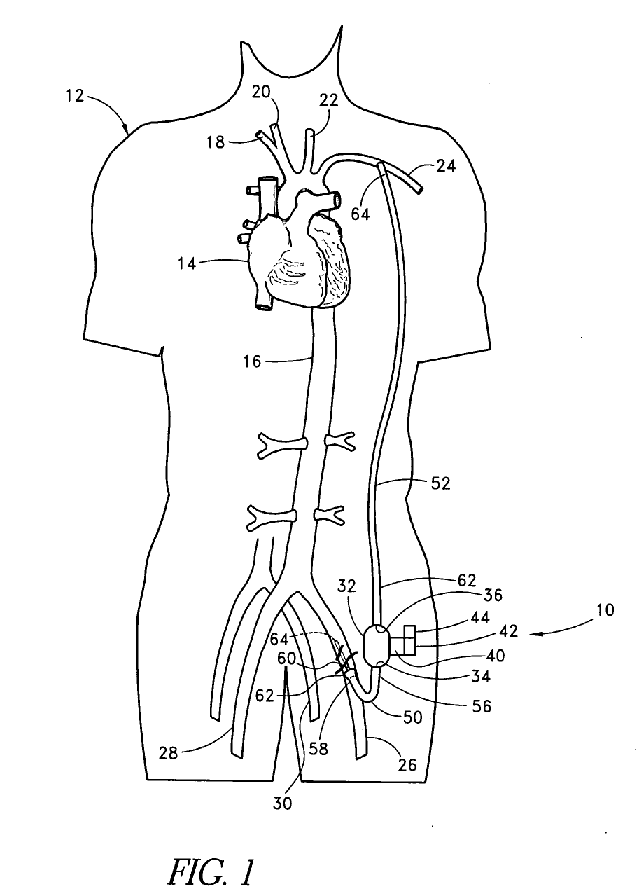

With reference to FIG. 1, a first embodiment of a heart assist system 10 is shown applied to a patient 12 having an ailing heart 14 and an aorta 16, from which peripheral brachiocephalic blood vessels extend, including the right subclavian artery 18, the right carotid artery 20, the left carotid artery 22, and the left subclavian artery 24. Extending from the descending aorta is another set of peripheral blood vessels, the left and right iliac arteries which transition into the left and right femoral arteries 26, 28, respectively. Each of the arteries 16, 18, 20, 22, 24, 26, and 28 generally conveys blood away from the heart. The vasculature includes a venous system that generally conveys blood to the heart. As discussed in more detail below, the heart assist systems described herein can also be applied to non-primary veins, including the left femoral vein 30.

The heart assist system 10 comprises a pump 32, having an inlet 34 and an outlet 36 for connection of conduits thereto. The pump 32 preferably is a rotary pump, either an axial type or a centrifugal type, although other types of pumps may be used, whether commercially-available or customized. The pump 32 preferably is sufficiently small to be implanted subcutaneously and preferably extrathoracically, for example in the groin area of the patient 12, without the need for major invasive surgery. Because the heart assist system 10 is an extracardiac system, no valves are necessary. Any inadvertent backflow through the pump 32 and/or through the inflow conduit would not harm the patient 12.

Regardless of the style or nature chosen, the pump 32 is sized to generate blood flow at subcardiac volumetric rates, less than about 50% of the flow rate of an average healthy heart, although flow rates above that may be effective. Thus, the pump 32 is sized and configured to discharge blood at volumetric flow rates anywhere in the range of 0.1 to 3 liters per minute, depending upon the application desired and/or the degree of need for heart assist. For example, for a patient experiencing advanced congestive heart failure, it may be preferable to employ a pump that has an average subcardiac rate of 2.5 to 3 liters per minute. In other patients, particularly those with minimal levels of heart failure, it may be preferable to employ a pump that has an average subcardiac rate of 0.5 liters per minute or less. In yet other patients it may be preferable to employ a pump that is a pressure wave generator that uses pressure to augment the flow of blood generated by the heart.

In one embodiment, the pump 32 is a continuous flow pump that superimposes continuous blood-flow on the pulsatile aortic blood-flow. In another embodiment, the pump 32 has the capability of synchronous actuation; i.e., it may be actuated in a pulsatile mode, either in copulsating or counterpulsating fashion.

For copulsating action, it is contemplated that the pump 32 would be actuated to discharge blood generally during systole, beginning actuation, for example, during isovolumic contraction before the aortic valve opens or as the aortic valve opens. The pump 32 would be static while the aortic valve is closed following systole, ceasing actuation, for example, when the aortic valve closes.

For counterpulsating actuation, it is contemplated that the pump 32 would be actuated generally during diastole, ceasing actuation, for example, before or during isovolumic contraction. Such an application would permit and/or enhance coronary blood perfusion. In this application, it is contemplated that the pump 32 would be static during the balance of systole after the aortic valve is opened, to lessen the burden against which the heart must pump. The aortic valve being open encompasses the periods of opening and closing, wherein blood is flowing therethrough.

It should be recognized that the designations copulsating and counterpulsating are general identifiers and are not limited to specific points in the patient's heart cycle when the pump 32 begins and discontinues actuation. Rather, they are intended to generally refer to pump actuation in which the pump 32 is actuating, at least in part, during systole and diastole, respectively. For example, it is contemplated that the pump 32 might be activated to be out of phase from true copulsating or counterpulsating actuation described herein, and still be synchronous, depending upon the specific needs of the patient or the desired outcome. One might shift actuation of the pump 32 to begin prior to or after isovolumic contraction or to begin before or after isovolumic relaxation.

Furthermore, the pulsatile pump may be actuated to pulsate asynchronously with the patient's heart. Typically, where the patient's heart is beating irregularly, there may be a desire to pulsate the pump 32 asynchronously so that the perfusion of blood by the heart assist system 10 is more regular and, thus, more effective at oxygenating the organs. Where the patient's heart beats regularly, but weakly, synchronous pulsation of the pump 32 may be preferred.

The pump 32 is driven by a motor 40 and/or other type of drive means and is controlled preferably by a programmable controller 42 that is capable of actuating the pump 32 in pulsatile fashion, where desired, and also of controlling the speed or output of the pump 32. For synchronous control, the patient's heart would preferably be monitored with an EKG in which feedback would be provided to the controller 42. The controller 42 is preferably programmed through external means, such as, for example, RF telemetry circuits of the type commonly used within implantable pacemakers and defibrillators. The controller may also be autoregulating to permit automatic regulation of the speed, and/or regulation of the synchronous or asynchronous pulsation of the pump 32, based upon feedback from ambient sensors monitoring parameters, such as pressure or the patient's EKG. It is also contemplated that a reverse-direction pump be used, if desired, in which the controller is capable of reversing the direction of either the drive means or the impellers of the pump. Such a pump might be used where it is desirable to have the option of reversing the direction of circulation between two blood vessels.

Power to the motor 40 and the controller 42 may be provided by a power source 44, such as a battery, that is preferably rechargeable by an external induction source (not shown), such as an RF induction coil that may be electromagnetically coupled to the battery to induce a charge therein. Alternative power sources are also possible, including a device that draws energy directly from the patient's body; e.g., the patient's muscles, chemicals or heat. The pump can be temporarily stopped during recharging with no appreciable life threatening effect, because the system only supplements the heart, rather than substituting for the heart.

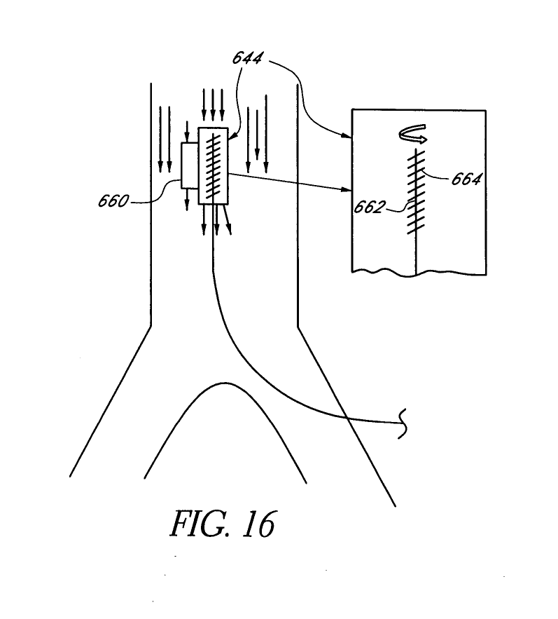

While the controller 42 and power source 44 are preferably pre-assembled to the pump 32 and implanted therewith, it is also contemplated that the pump 32 and motor 40 be implanted at one location and the controller 42 and the power source 44 be implanted in a separate location. In one alternative arrangement, the pump 32 may be driven externally through a percutaneous drive line or cable, as shown in FIG. 16. In another variation, the pump, motor and controller may be implanted and powered by an extracorporeal power source. In the latter case, the power source could be attached to the side of the patient to permit fully ambulatory movement.

The inlet 34 of the pump 32 is preferably connected to an inflow conduit 50 and an outflow conduit 52 to direct blood flow from one peripheral blood vessel to another. The conduits 50, 52 preferably are flexible conduits, as discussed more fully below. The conduits 50, 52 are coupled with the peripheral vessels in different ways in various embodiments of the heart assist system 10. As discussed more fully below, at least one of the conduits 50, 52 can be connected to a peripheral vessel, e.g., as a graft, using an anastomosis connection, and at least one of the conduits 50, 52 can be coupled with the same or another vessel via insertion of a cannula into the vasculature. Also, more than two conduits are used in some embodiments, as discussed below.

The inflow and outflow conduits 50, 52 may be formed from Dacron, Hemashield, Gortex, PVC, polyurethane, PTFE, ePTFE, nylon, or PEBAX materials, although other synthetic materials may be suitable. The inflow and outflow conduits 50, 52 may also comprise biologic materials or pseudobiological (hybrid) materials (e.g., biologic tissue supported on a synthetic scaffold). The inflow and outflow conduits 50, 52 are preferably configured to minimize kinks so blood flow is not meaningfully interrupted by normal movements of the patient or compressed easily from external forces. In some cases, the inflow and/or outflow conduits 50, 52 may come commercially already attached to the pump 32. Where it is desired to implant the pump 32 and the conduits 50, 52, it is preferable that the inner diameter of the conduits 50, 52 be less than 25 mm, although diameters slightly larger may be effective.

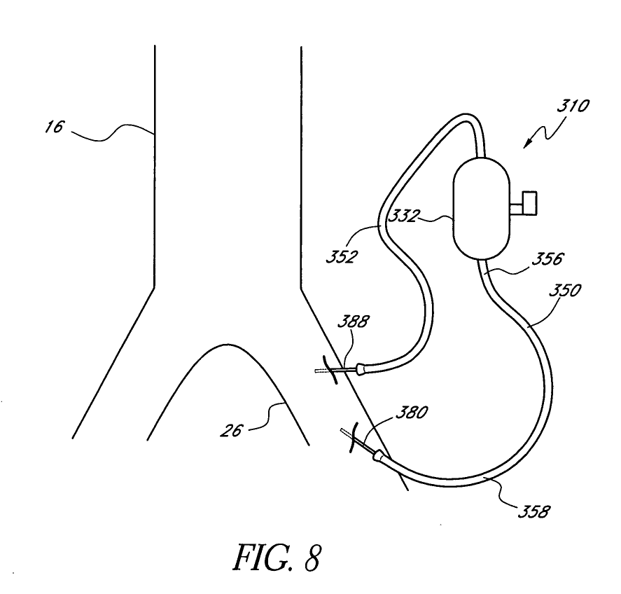

In one preferred application, the heart assist system 10 is applied in an arterial-arterial fashion; for example, as a femoral-axillary connection, as is shown in FIG. 1. Those of skill in the art will appreciate that an axillary-femoral connection would also be effective using the embodiments described herein. Indeed, those of skill in the art will appreciate that the heart assist system 10 might be applied to any of the peripheral blood vessels in the patient. Another application of the heart assist system 10 couples the conduits 50, 52 with the same non-primary vessel in a manner similar to the application shown in FIG. 8 and discussed below.

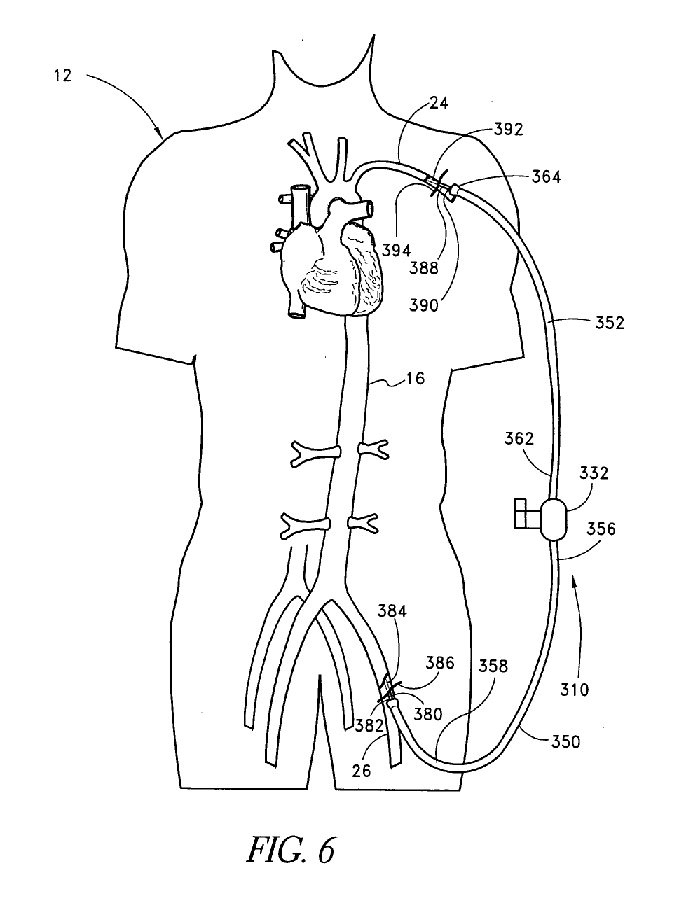

FIG. 1 shows that the inflow conduit 50 has a first end 56 that connects with the inlet 34 of the pump 32 and a second end 58 that is coupled with a first non-primary blood vessel (e.g., the left femoral artery 26) by way of an inflow cannula 60. The inflow cannula 60 has a first end 62 and a second end 64. The first end 62 is sealably connected to the second end 58 of the inflow conduit 50. The second end 64 is inserted into the blood vessel (e.g., the left femoral artery 26). Although shown as discrete structures in FIG. 1, those of skill in the art will appreciate that the inflow conduit 50 and the cannula 60 may be unitary in construction. The cannula 60 can take any suitable form, e.g., defining a lumen with an inner size that increases distally.

Where the conduit 50 is at least partially extracorporeal, the inflow cannula 60 also may be inserted through a surgical opening (e.g., as shown in FIG. 6 and described in connection therewith) or percutaneously, with or without an introducer sheath (not shown). In other applications, the inflow cannula 60 could be inserted into the right femoral artery or any other peripheral artery.

FIG. 1 shows that the outflow conduit 52 has a first end 66 that connects to the outlet 36 of the pump 32 and a second end 68 that connects with a second peripheral blood vessel, preferably the left subclavian artery 24 of the patient 12, although the right axillary artery, or any other peripheral artery, would be acceptable. In one application, the connection between the outflow conduit 52 and the second blood vessel is via an end-to-side anastomosis, although a side-to-side anastomosis connection might be used mid-stream of the conduit where the outflow conduit were connected at its second end to yet another blood vessel or at another location on the same blood vessel (neither shown). Preferably, the outflow conduit 52 is attached to the second blood vessel at an angle that results in the predominant flow of blood out of the pump 32 proximally toward the aorta 16 and the heart 14, such as is shown in FIG. 1, while still maintaining sufficient flow distally toward the hand to prevent limb ischemia.

In another embodiment, the inflow conduit 50 is connected to the first blood vessel via an end-to-side anastomosis, rather than via the inflow cannula 60. The inflow conduit 50 could also be coupled with the first blood vessel via a side-to-side anastomosis connection mid-stream of the conduit, where the inflow conduit connects at its second end to an additional blood vessel or at another location on the same blood vessel (neither shown). Further details of these arrangements and other related applications are described in U.S. application Ser. No. 10/289,467, filed Nov. 6, 2002, the entire contents of which is hereby incorporated by reference in its entirety and made a part of this specification.

In another embodiment, the outflow conduit 52 also is coupled with the second blood vessel via a cannula, as shown in FIG. 6. This connection may be achieved in a manner similar to that shown in FIG. 1 in connection with the first blood vessel.

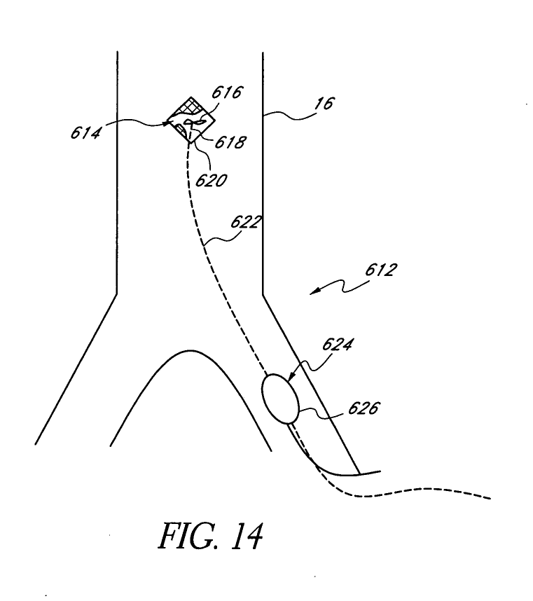

Preferably, the heart assist system 10 is applied to the peripheral or non-primary blood vessels subcutaneously; e.g., at a shallow depth just below the skin or first muscle layer so as to avoid major invasive surgery. It is also preferred that the heart assist system 10 be applied extrathoracically to avoid the need to invade the patient's chest cavity. Where desired, the entire heart assist system 10 may be implanted within the patient 12, either extravascularly, e.g., as in FIG. 1, or at least partially intravascularly, e.g., as in FIGS. 14-16.

In the case of an extravascular application, the pump 32 may be implanted, for example, into the groin area, with the inflow conduit 50 fluidly connected subcutaneously to, for example, the femoral artery 26 proximate the pump 32. The outflow conduit would be tunneled subcutaneously through to, for example, the left subclavian artery 24. In an alternative arrangement, the pump 32 and associated drive and controller could be temporarily fastened to the exterior skin of the patient, with the inflow and outflow conduits 50, 52 connected percutaneously. In either case, the patient may be ambulatory without restriction of tethered lines.

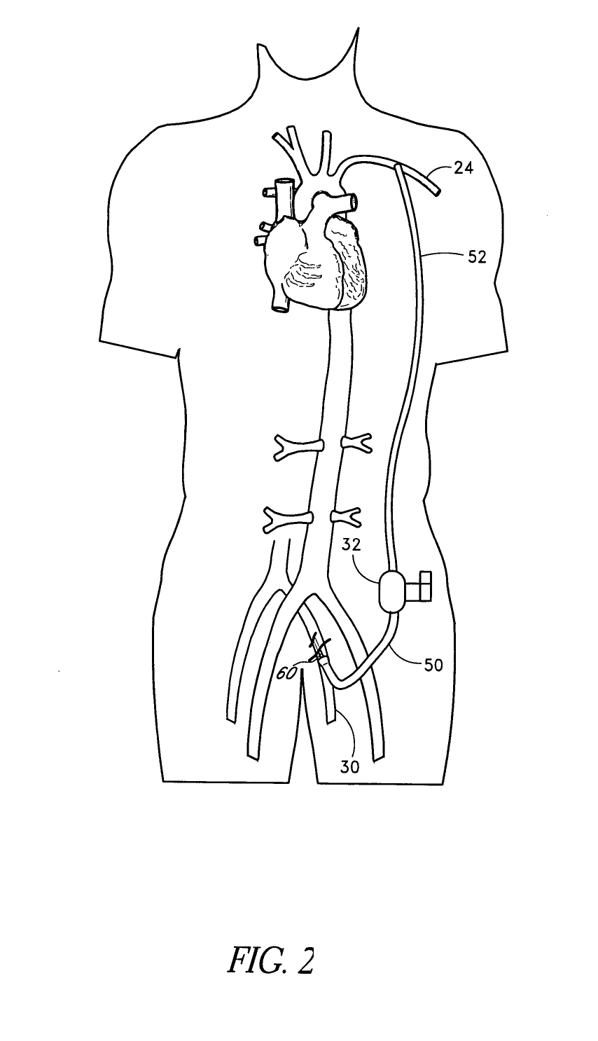

While the heart assist system 10 and other heart assist systems described herein may be applied to create an arterial-arterial flow path, given the nature of the heart assist systems, i.e., supplementation of circulation to meet organ demand, a venous-arterial flow path may also be used. For example, with reference to FIG. 2, one application of the heart assist system 10 couples the inflow conduit 50 with a non-primary vein of the patient 12, such as the left femoral vein 30. In this arrangement, the outflow conduit 50 may be fluidly coupled with one of the peripheral arteries, such as the left subclavian artery 24. Arterial-venous arrangements are contemplated as well.

In those venous-arterial cases where the inflow is connected to a vein and the outflow is connected to an artery, the pump 32 is preferably sized to permit flow sufficiently small so that oxygen-deficient blood does not rise to unacceptable levels in the arteries. The connections to the non-primary veins could be by one or more of the approaches described above for connecting to a non-primary artery. The extracardiac assist system could also be applied as a venous-venous flow path, wherein the inflow and outflow are connected to separate peripheral veins. In addition, an alternative embodiment comprises two discrete pumps and conduit arrangements, one being applied as a venous-venous flow path, and the other as an arterial-arterial flow path.

When venous blood is mixed with arterial blood, either at the inlet of the pump or at the outlet of the pump, the ratio of venous blood to arterial blood is preferably controlled to maintain an arterial saturation of a minimum of 80% at the pump inlet or outlet. Arterial saturation can be measured and/or monitored by pulse oximetry, laser doppler, colorimetry or other methods used to monitor blood oxygen saturation. The venous blood flow into the system can then be controlled by regulating the amount of blood allowed to pass through the conduit from the venous-side connection.

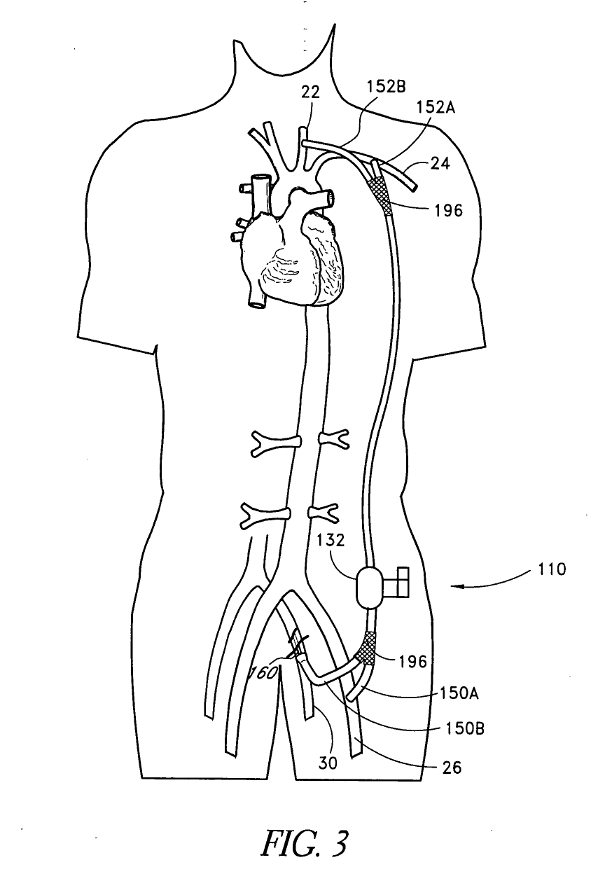

FIG. 3 shows another embodiment of a heart assist system 110 applied to the patient 12. For example, the heart assist system 110 includes a pump 132 in fluid communication with a plurality of inflow conduits 150A, 150B and a plurality of outflow conduits 152A, 152B. Each pair of conduits converge at a generally Y-shaped convergence 196 that converge the flow at the inflow end and diverge the flow at the outflow end. Each conduit may be connected to a separate peripheral blood vessel, although it is possible to have two connections to the same blood vessel at remote locations.

In one arrangement, all four conduits are connected to peripheral arteries. In another arrangement, one or more of the conduits could be connected to veins. In the arrangement of FIG. 3, the inflow conduit 150A is connected to the left femoral artery 26 while the inflow conduit 150B is connected to the left femoral vein 30. The outflow conduit 152A is connected to the left subclavian artery 24 while the outflow conduit 152B is connected to the left carotid artery 22. Preferably at least one of the conduits 150A, 150B, 152A, and 152B is coupled with a corresponding vessel via a cannula. In the illustrated embodiment, the inflow conduit 150B is coupled with the left femoral vein 30 via a cannula 160. The cannula 160 is coupled in a manner similar to that shown in FIG. 2 and described in connection with the cannula 60. The cannula 160 can take suitable form, e.g., defining a lumen with an inner size that increases distally as discussed below in connection with FIGS. 17-27.

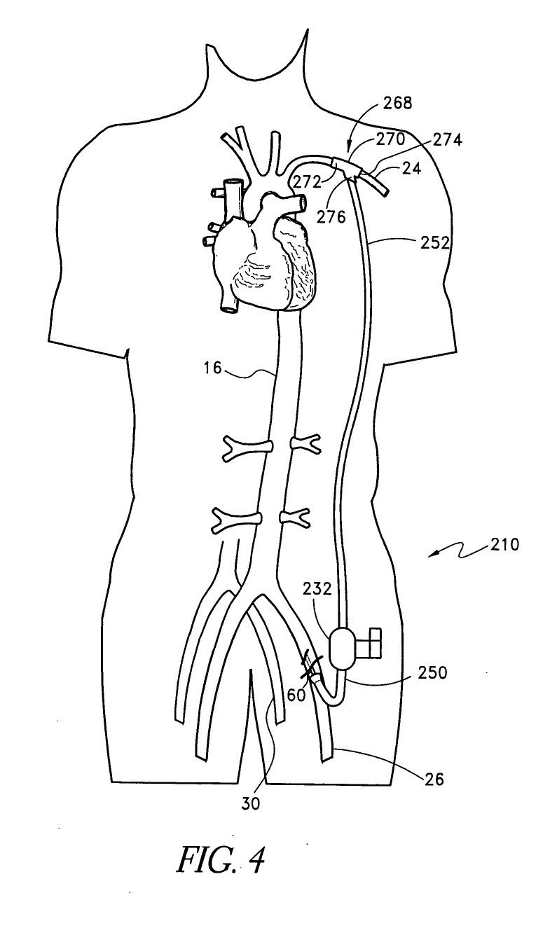

The connections of any or all of the conduits of the system 110 to the blood vessels may be via an anastomosis connection or via a connector, as described below in connection with FIG. 4. In addition, the embodiment of FIG. 3 may be applied to any combination of peripheral blood vessels that would best suit the patient's condition. For example, it may be desired to have one inflow conduit and two outflow conduits or vice versa. More than two conduits may be used on the inflow or outflow side, where the number of inflow conduits is not necessarily equal to the number of outflow conduits.

Where an anastomosis connection is not desired, a connector may be used to connect at least one of the inflow conduit and the outflow conduit to a peripheral blood vessel. With reference to FIG. 4, an embodiment of a heart assist system 210 is shown, wherein an outflow conduit 252 is connected to a non-primary blood vessel, e.g., the left subclavian artery 24, via a connector 268 that comprises a three-opening fitting. In one embodiment, the connector 268 comprises an intra-vascular, generally T-shaped fitting 270 having a proximal end 272 (relative to the flow of blood in the left axillary artery and therethrough), a distal end 274, and an angled divergence 276 permitting connection to the outflow conduit 252 and the left subclavian artery 24. The proximal and distal ends 274, 276 of the fittings 272 permit connection to the blood vessel into which the fitting is positioned, e.g., the left subclavian artery 24. The angle of divergence 276 of the fittings 272 may be 90 degrees or less in either direction from the axis of flow through the blood vessel, as optimally selected to generate the needed flow distally toward the hand to prevent limb ischemia, and to insure sufficient flow and pressure toward the aorta to provide the circulatory assistance and workload reduction needed while minimizing or avoiding endothelial damage to the blood vessel. In another embodiment, the connector 268 is a sleeve (not shown) that surrounds and attaches to the outside of the non-primary blood vessel where, within the interior of the sleeve, a port to the blood vessel is provided to permit blood flow from the outflow conduit 252 when the conduit 252 is connected to the connector 268.

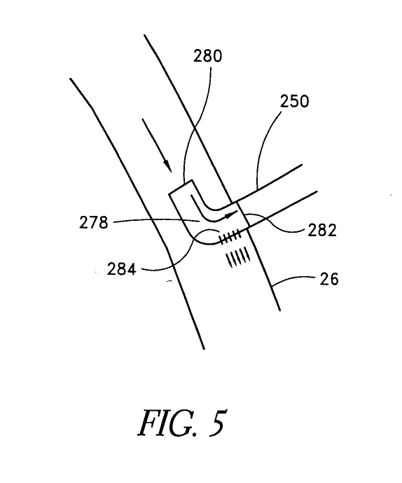

Other types of connectors having other configurations are contemplated that may avoid the need for an anastomosis connection or that permit connection of the conduit(s) to the blood vessel(s). For example, it is contemplated that an L-shaped connector be used if it is desired to withdraw blood more predominantly from one direction of a peripheral vessel or to direct blood more predominantly into a peripheral vessel. Referring to FIG. 5, the inflow conduit 250 is fluidly connected to a peripheral vessel, for example, the left femoral artery 26, using an L-shaped connector 278. Of course the system 210 could be configured so that the outflow conduit 252 is coupled to a non-primary vessel via the L-shaped connector 278 and the inflow conduit 250 is coupled via a cannula, as shown in FIG. 3.

The L-shaped connector 278 has an inlet port 280 at a proximal end and an outlet port 282 through which blood flows into the inflow conduit 250. The L-shaped connector 278 also has an arrangement of holes 284 within a wall positioned at a distal end opposite the inlet port 280 so that some of the flow drawn into the L-shaped connector 278 is diverted through the holes 284, particularly downstream of the L-shaped connector 278, as in this application. A single hole 284 in the wall could also be effective, depending upon size and placement. The L-shaped connector 278 may be a deformable L-shaped catheter percutaneously applied to the blood vessel or, in an alternative embodiment, be connected directly to the walls of the blood vessel for more long term application. By directing some blood flow downstream of the L-shaped connector 278 during withdrawal of blood from the vessel, ischemic damage downstream from the connector may be avoided. Such ischemic damage might otherwise occur if the majority of the blood flowing into the L-shaped connector 278 were diverted from the blood vessel into the inflow conduit 252. It is also contemplated that a connection to the blood vessels might be made via a cannula, wherein the cannula is implanted, along with the inflow and outflow conduits.

One advantage of discrete connectors manifests in their application to patients with chronic CHF. A connector eliminates a need for an anastomosis connection between the conduits 250, 252 and the peripheral blood vessels where it is desired to remove and/or replace the system more than one time. The connectors could be applied to the first and second blood vessels semi-permanently, with an end cap applied to the divergence for later quick-connection of the cardiac assist system to the patient. In this regard, a patient might experience the benefit of the heart assist systems described herein periodically, without having to reconnect and redisconnect the conduits 250, 252 from the blood vessels via an anastomosis procedure each time. Each time it is desired to implement any of the embodiments of the heart assist system, the end caps would be removed and a conduit attached to the connector(s) quickly.

In the preferred embodiment of the connector 268, the divergence 276 is oriented at an acute angle significantly less than 90 degrees from the axis of the T-shaped fitting 270, as shown in FIG. 4, so that a majority of the blood flowing through the outflow conduit 252 into the blood vessel (e.g., left subclavian artery 24) flows in a direction proximally toward the heart 14, rather than in the distal direction. In an alternative embodiment, the proximal end 272 of the T-shaped fitting 270 may have a diameter larger than the diameter of the distal end 274, without need of having an angled divergence, to achieve the same result.

With or without a connector, with blood flow directed proximally toward the aorta 16, the result may be concurrent flow down the descending aorta, which will result in the reduction of afterload, impedence, and/or reducing left ventricular end diastolic pressure and volume (preload). Thus, the heart assist systems described herein may be applied so to reduce the afterload on the patient's heart, permitting at least partial if not complete CHF recovery, while supplementing blood circulation. Concurrent flow depends upon the phase of operation of the pulsatile pump and the choice of second blood vessel to which the outflow conduit is connected.

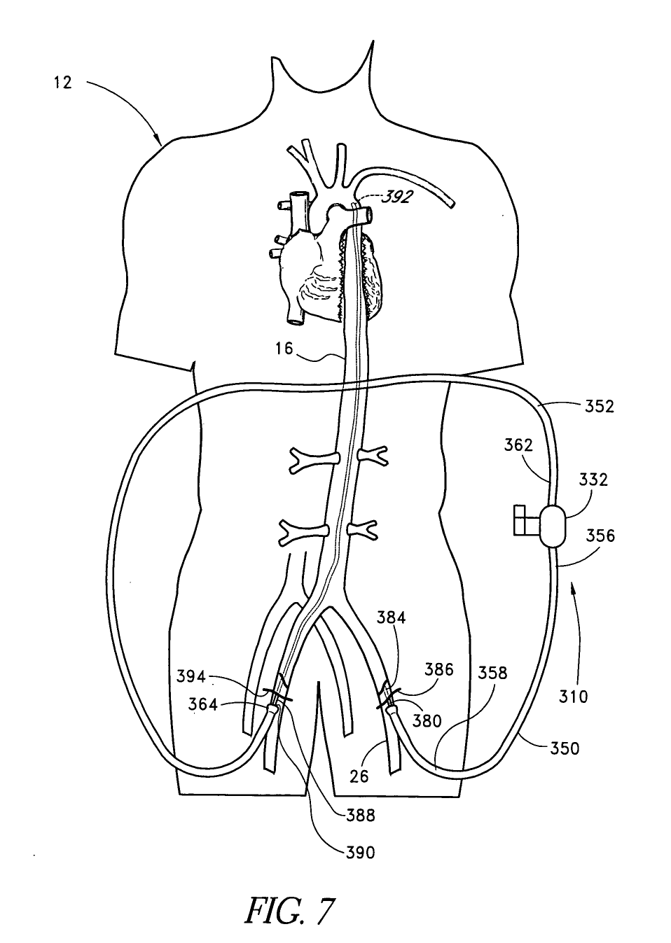

A partial external application of the heart assist systems may be appropriate where a patient with heart failure is suffering an acute decompensation episode; i.e., is not expected to survive long, or in the earlier stages of heart failure (where the patient is in New York Heart Association Classification (NYHAC) functional classes II or III). With reference to FIGS. 6 and 7, another embodiment of a heart assist system 310 is applied percutaneously to a patient 312 to connect two non-primary blood vessels wherein a pump 332 and its associated driving means and controls are employed extracorporeally.

The pump 332 has an inflow conduit 350 and an outflow conduit 352 associated therewith for connection to two non-primary blood vessels. The inflow conduit 350 has a first end 356 and a second end 358 wherein the second end 358 is connected to a first non-primary blood vessel (e.g., femoral artery 26) by way of an inflow cannula 380. The inflow cannula 380 has a first end 382 sealably connected to the second end 358 of the inflow conduit 350. The inflow cannula 380 also has a second end 384 that is inserted through a surgical opening 386 or an introducer sheath (not shown) and into the blood vessel (e.g., the left femoral artery 26).

Similarly, the outflow conduit 352 has a first end 362 and a second end 364 wherein the second end 364 is connected to a second non-primary blood vessel (e.g., the left subclavian artery 24, as shown in FIG. 6, or the right femoral artery 28, as shown in FIG. 7) by way of an outflow cannula 388. Like the inflow cannula 380, the outflow cannula 388 has a first end 390 sealably connected to the second end 364 of the outflow conduit 352. The outflow cannula 388 also has a second end 392 that is inserted through surgical opening 394 or an introducer sheath (not shown) and into the second blood vessel (e.g., the left subclavian artery 24 or the right femoral artery 28). The cannulae 380 and 388 can take any suitable form, e.g., defining a lumen with an inner size that increases distally.

As shown in FIG. 7, the second end 392 of the outflow cannula 388 may extend well into the aorta 16 of the patient 12, for example, proximal to the left subclavian artery. If desired, it may also terminate within the left subclavian artery or the left axillary artery, or in other blood vessels, such as the mesenteric or renal arteries (not shown), where in either case, the outflow cannula 388 has passed through at least a portion of a primary artery (in this case, the aorta 16). Also, if desired, blood drawn into the extracardiac system 310 described herein may originate from the descending aorta (or an artery branching therefrom) and be directed into a blood vessel that is neither the aorta nor pulmonary artery. By use of a percutaneous application, the heart assist system 310 may be applied temporarily without the need to implant any aspect thereof or to make anastomosis connections to the blood vessels.

An alternative variation of the embodiment of FIG. 6 may be used where it is desired to treat a patient periodically, but for short periods of time each occasion and without the use of special connectors. With this variation, the second ends of the inflow and outflow conduits 350, 352 may be more permanently connected to the associated blood vessels via, for example, an anastomosis connection, wherein a portion of each conduit proximate to the blood vessel connection is implanted percutaneously with a removable cap enclosing the externally-exposed first end (or an intervening end thereof) of the conduit external to the patient. When it is desired to provide a circulatory flow path to supplement blood flow, the removable cap on each exposed percutaneously-positioned conduit could be removed and the pump (or the pump with a length of inflow and/or outflow conduit attached thereto) inserted between the exposed percutaneous conduits. In this regard, a patient may experience the benefit of the cardiac assist system periodically, without having to reconnect and redisconnect the conduits from the blood vessels each time.

Specific methods of applying this alternative embodiment may further comprise coupling the inflow conduit 352 upstream of the outflow conduit 350 (as shown in FIG. 8), although the reverse arrangement is also contemplated. It is also contemplated that either the cannula 380 coupled with the inflow conduit 350 or the cannula 388 coupled with the outflow conduit 352 may extend through the non-primary blood vessel to a second blood vessel (e.g., through the left femoral artery 26 to the aorta 16 proximate the renal branch) so that blood may be directed from the non-primary blood vessel to the second blood vessel or vice versa.

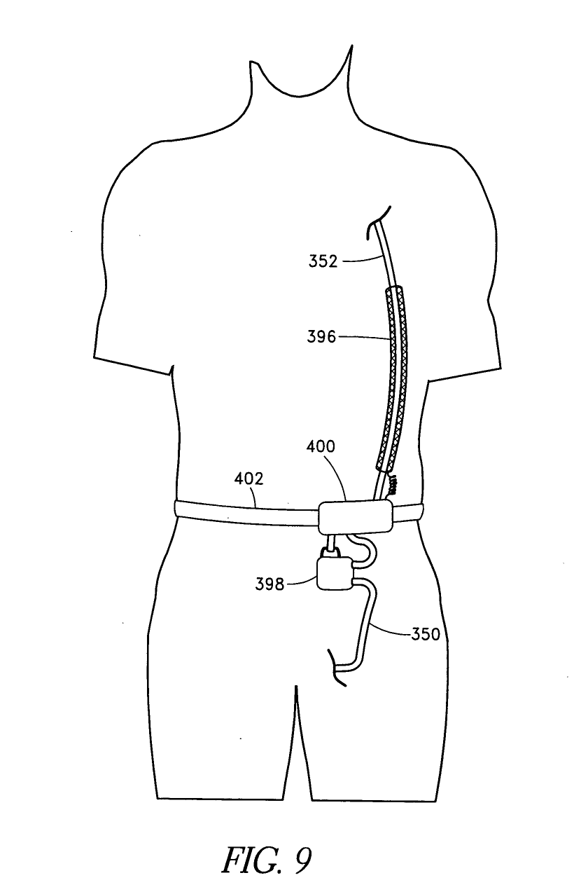

A means for minimizing the loss of thermal energy from the patient's blood may be provided where any of the heart assist systems described herein are applied extracorporeally. Such means for minimizing the loss of thermal energy may comprise, for example, a heated bath through which the inflow and outflow conduits pass or, alternatively, thermal elements secured to the exterior of the inflow and outflow conduits. Referring to FIG. 9, one embodiment comprises an insulating wrap 396 surrounding the outflow conduit 352 having one or more thermal elements passing therethrough. The elements may be powered, for example, by a battery (not shown). One advantage of thermal elements is that the patient may be ambulatory, if desired. Other means that are known by persons of ordinary skill in the art for ensuring that the temperature of the patient's blood remains at acceptable levels while traveling extracorporeally are also contemplated.

If desired, the cardiac assist system may further comprise a reservoir that is either contained within or in fluid communication with the inflow conduit. This reservoir is preferably made of materials that are nonthrombogenic. Referring to FIG. 9, a reservoir 398 is positioned fluidly in line with the inflow conduit 350. The reservoir 398 serves to sustain adequate blood in the system when the pump demand exceeds momentarily the volume of blood available in the peripheral blood vessel in which the inflow conduit resides until the pump output can be adjusted. The reservoir 398 reduces the risk of excessive drainage of blood from the peripheral blood vessel, which may occur when cardiac output falls farther than the already diminished baseline level of cardiac output, or when there is systemic vasodilation, as can occur, for example, with septic shock. It is contemplated that the reservoir 398 would be primed with an acceptable solution, such as saline, when the present system is first applied to the patient.

As explained above, one of the advantages of several embodiments of the heart assist system is that such systems permit the patient to be ambulatory. The systems may be designed to be portable, so the patient may carry the system. Referring to FIG. 9, the system may include a portable case 400 with a belt strap 402 to house the pump, power supply and/or the controller, along with certain portions of the inflow and/or outflow conduits, if necessary. Alternatively, or in addition, the system may include a shoulder strap, a backpack, a fanny pack, or other apparatus that permits effective portability. As shown in FIG. 9, blood is drawn through the inflow conduit 350 into a pump contained within the portable case 400, where it is discharged into the outflow conduit 352 back into the patient.

B. Heart Assist Systems and Methods Employing Single-Site Application

As discussed above, heart assist systems can be applied to a patient through a single cannulation site. Such single-site systems can be configured with a pump located outside the vasculature of a patient, e.g., as extravascular pumping systems, inside the vasculature of the patient, e.g., as intravascular systems, or a hybrid thereof, e.g., partially inside and partially outside the vasculature of the patient.

1. Single-Site Application of Extravascular Pumping Systems

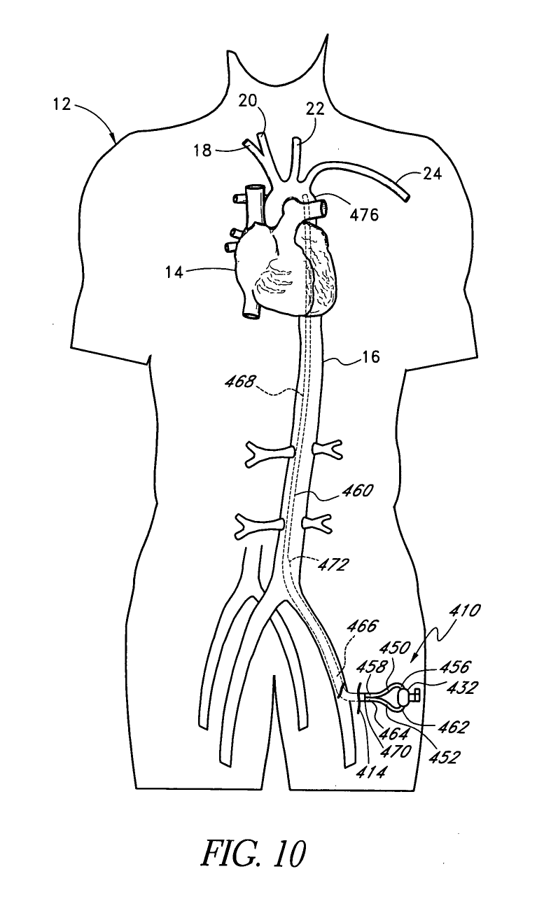

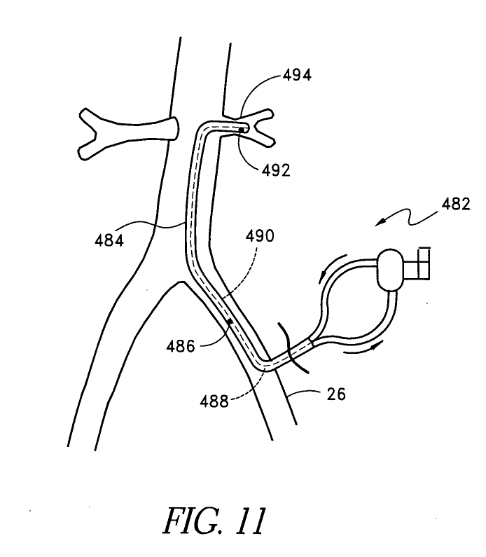

FIGS. 10 and 11 illustrate extracardiac heart assist systems that employ an extravascular pump and that can be applied as a single-site system. FIG. 10 shows a system 410 that is applied to a patient 12 through a single cannulation site 414 while inflow and outflow conduits fluidly communicate with non-primary vessels. The heart assist system 410 is applied to the patient 12 percutaneously through a single-site to couple two blood vessels with a pump 432. The pump 432 can have any of the features described above in connection with the pump 32. The pump 432 has an inflow conduit 450 and an outflow conduit 452 associated therewith. The inflow conduit 450 has a first end 456 and a second end 458. The first end 456 of the inflow conduit 450 is connected to the inlet of the pump 432 and the second end 458 of the inflow conduit 450 is fluidly coupled with a first non-primary blood vessel (e.g., the femoral artery 26) by way of a multilumen cannula 460. Similarly, the outflow conduit 452 has a first end 462 and a second end 464. The first end 462 of the outflow conduit 452 is connected to the outlet of the pump 432 and the second end 464 of the outflow conduit 452 is fluidly coupled with a second blood vessel (e.g., the descending aorta 16) by way of the multilumen cannula 460.

In one embodiment, the multilumen cannula 460 includes a first lumen 466 and a second lumen 468. The first lumen 466 extends from a proximal end 470 of the multilumen cannula 460 to a first distal end 472. The second lumen 468 extends from the proximal end 470 to a second distal end 474. In the illustrated embodiment, the second end 458 of the inflow conduit 450 is connected to the first lumen 466 of the multilumen cannula 460 and the second end 464 of the outflow conduit 452 is connected to the second lumen 468 of the multilumen cannula 460.

Where there is a desire for the patient 12 to be ambulatory, the multilumen cannula 460 preferably is made of material sufficiently flexible and resilient to permit the patient 12 to comfortably move about while the multilumen cannula 460 is indwelling in the patient's blood vessels without causing any vascular trauma.

The application shown in FIG. 10 and described above results in flow from the first distal end 472 to the second distal end 474. Of course, the flow direction may be reversed using the same arrangement, resulting in flow from the second distal end 474 to the first distal end 472. In some applications, the system 410 is applied in an arterial-arterial fashion. For example, as illustrated, the multilumen cannula 460 can be inserted into the left femoral artery 26 of the patient 12 and guided superiorly through the descending aorta to one of numerous locations. In one application, the multilumen cannula 460 can be advanced until the distal end 474 is located in the aortic arch 476 of the patient 12. The blood could discharge, for example, directly into the descending aorta proximate an arterial branch, such as the left subclavian artery or directly into the peripheral mesenteric artery (not shown).

The pump 432 draws blood from the patient's vascular system in the area near the distal end 472 and into the lumen 466. This blood is further drawn into the lumen of the conduit 450 and into the pump 432. The pump 432 then expels the blood into the lumen of the outflow conduit 452, which carries the blood into the lumen 468 of the multilumen cannula 460 and back into the patient's vascular system in the area near the distal end 474.

FIG. 11 shows another embodiment of a heart assist system 482 that is similar to the heart assist system 410, except as set forth below. The system 482 employs a multilumen cannula 484. In one application, the multilumen cannula 484 is inserted into the left femoral artery 26 and guided superiorly through the descending aorta to one of numerous locations. Preferably, the multilumen cannula 484 has an inflow port 486 that is positioned in one application within the left femoral artery 26 when the cannula 484 is fully inserted so that blood drawn from the left femoral artery 26 is directed through the inflow port 486 into a first lumen 488 in the cannula 484. The inflow port 486 can also be positioned in any other suitable location within the vasculature, described herein or apparent to one skilled in the art. This blood is then pumped through a second lumen 490 in the cannula 484 and out through an outflow port 492 at the distal end of the cannula 484.

The outflow port 492 may be situated within, for example, a mesenteric artery 494 such that blood flow results from the left femoral artery 26 to the mesenteric artery 494. The blood could discharge, for example, directly into the descending aorta proximate an arterial branch, such as the renal arteries, the left subclavian artery, or directly into the peripheral mesenteric artery 494, as illustrated in FIG. 11. Where there is a desire for the patient to be ambulatory, the multilumen cannula 484 preferably is made of material sufficiently flexible and resilient to permit the patient 12 to comfortably move about while the cannula 484 is indwelling in the patient's blood vessels without causing any vascular trauma.

Additional details of the multilumen cannula 460 may be found in U.S. patent application Ser. No. 10/078,283, filed Feb. 14, 2002, entitled A MULTILUMEN CATHETER FOR MINIMIZING LIMB ISCHEMIA and in U.S. patent application Ser. No. 10/706,346, filed Nov. 12, 2003, entitled CANNULAE HAVING REDIRECTING TIP, which are hereby expressly incorporated by reference in their entirety and made a part of this specification.

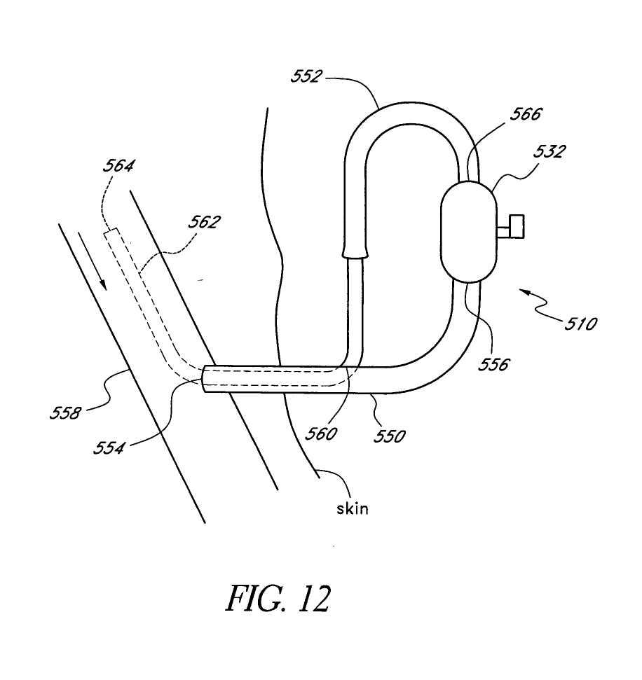

FIG. 12 shows another heart assist system 510 that takes further advantage of supplemental blood perfusion and heart load reduction benefits while remaining minimally invasive in application. The heart assist system 510 is an extracardiac pumping system that includes a pump 532, an inflow conduit 550 and an outflow conduit 552. In the illustrated embodiment, the inflow conduit 550 comprises a vascular graft. The vascular graft conduit 550 and the outflow conduit 552 are fluidly coupled to a pump 532. The pump 532 is configured to pump blood through the patient at subcardiac volumetric rates, and has an average flow rate that, during normal operation thereof, is substantially below that of the patient's heart when healthy. In one variation, the pump 532 may be a rotary pump. Other pumps described herein, or any other suitable pump can also be used in the extracardiac pumping system 510. In one application, the pump 532 is configured so as to be implantable.

The vascular graft 550 has a first end 554 and a second end 556. The first end 554 is sized and configured to couple to a non-primary blood vessel 558 subcutaneously to permit application of the extracardiac pumping system 510 in a minimally-invasive procedure. In one application, the vascular graft conduit 550 is configured to couple to the blood vessel 558 via an anastomosis connection.

The second end 556 of the vascular graft 550 is fluidly coupled to the pump 532 to conduct blood between the non-primary blood vessel 558 and the pump 532. In the embodiment shown, the second end 556 is directly connected to the pump 532, but, as discussed above in connection with other embodiments, intervening fluid conducting elements may be interposed between the second end 556 of the vascular graft 550 and the pump 532. Examples of arrangements of vascular graft conduits may be found in U.S. application Ser. No. 09/780,083, filed Feb. 9, 2001, entitled EXTRA-CORPOREAL VASCULAR CONDUIT, which is hereby incorporated by reference in its entirety and made a part of this specification.

FIG. 12 illustrates that the present inventive embodiment further comprises means for coupling the outflow conduit 552 to the vascular graft 550, which may comprise in one embodiment an insertion site 560. In the illustrated embodiment, the insertion site 560 is located between the first end 554 and the second end 556 of the vascular graft 550. The outflow conduit 552 preferably is coupled with a cannula 562. The cannula 562 can take any suitable form, e.g., defining a lumen with an inner size that increases distally.

In the illustrated embodiment, the insertion site 560 is configured to receive the cannula 562 therethrough in a sealable manner. In another embodiment, the insertion site 560 is configured to receive the outflow conduit 552 directly. The cannula 562 includes a first end 564 sized and configured to be inserted through the insertion site 560, through the cannula 550, and through the non-primary blood vessel 558. The conduit 552 has a second end 566 fluidly coupled to the pump 532 to conduct blood between the pump 532 and the blood vessel 558.

The extracardiac pumping system 510 can be applied to a patient, as shown in FIG. 12, so that the outflow conduit 552 provides fluid communication between the pump 532 and a location upstream or downstream of the location where the cannula 562 enters the non-primary blood vessel 558. In another application, the cannula 562 is directed through the blood vessel to a different blood vessel, upstream or downstream thereof. Although the vascular graft 550 is described above as an “inflow conduit” and the conduit 552 is described above as an “outflow conduit,” in another application of this embodiment, the blood flow through the pumping system 510 is reversed (i.e., the pump 532 pumps blood in the opposite direction), whereby the vascular graft 550 is an outflow conduit and the conduit 552 is an inflow conduit.

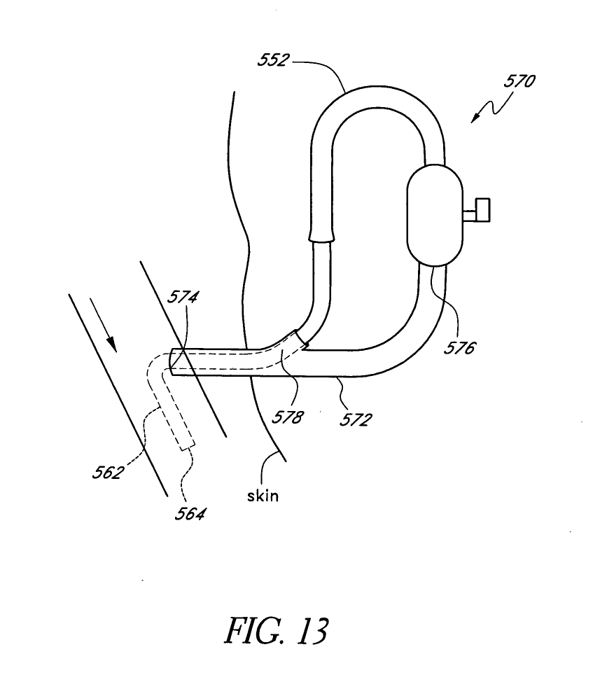

FIG. 13 shows a variation of the extracardiac pumping system shown in FIG. 12. In particular, a heart assist system 570 includes an inflow conduit 572 that comprises a first end 574, a second end 576, and means for connecting the outflow conduit 552 to the inflow conduit 572. In one embodiment, the inflow conduit 572 comprises a vascular graft. The extracardiac pumping system 570 is otherwise similar to the extracardiac pumping system 510. The means for connecting the conduit 552 to the inflow conduit 572 may comprise a branched portion 578. In one embodiment, the branched portion 578 is located between the first end 574 and the second end 576. The branched portion 578 is configured to sealably receive the distal end 564 of the outflow conduit 552. Where, as shown, the first end 564 of the outflow conduit 552 comprises the cannula 562, the branched portion 578 is configured to receive the cannula 562. The inflow conduit 572 of this arrangement comprises in part a multilumen cannula, where the internal lumen extends into the blood vessel 558. Other multilumen catheter arrangements are shown in U.S. application Ser. No. 10/078,283, incorporated by reference herein above.

2; Single-Site Application of Intravascular Pumping Systems

FIGS. 14-16 illustrate extracardiac heart assist systems that employ intravascular pumping systems. Such systems take further advantage of the supplemental blood perfusion and heart load reduction benefits discussed above while remaining minimally invasive in application. Specifically, it is contemplated to provide an extracardiac pumping system that comprises a pump that is sized and configured to be at least partially implanted intravascularly in any location desirable to achieve those benefits, while being insertable through a non-primary vessel.

FIG. 14 shows a heart assist system 612 that includes a pumping means 614 comprising preferably one or more rotatable impeller blades 616, although other types of pumping means 614 are contemplated, such as an Archimedes screw, a worm pump, or other means by which blood may be directed axially along the pumping means from a location upstream of an inlet to the pumping means to a location downstream of an outlet from the pumping means. Where one or more impeller blades 616 are used, such as in a rotary pump, such impeller blades 616 may be supported helically or otherwise on a shaft 618 within a housing 620. The housing 620 may be open, as shown, in which the walls of the housing 620 are open to blood flow therethrough. The housing 620 may be entirely closed, if desired, except for an inlet and outlet (not shown) to permit blood flow therethrough in a more channeled fashion. For example, the housing 620 could be coupled with or replaced by a cannula with a lumen that has an inner size that increases distally. The heart assist system 612 serves to supplement the kinetic energy of the blood flow through the blood vessel in which the pump is positioned, e.g., the aorta 16.

The impeller blade(s) 616 of the pumping means 614 of this embodiment may be driven in one of a number of ways known to persons of ordinary skill in the art. In the embodiment shown in FIG. 14, the impeller blade(s) 616 are driven mechanically via a rotatable cable or drive wire 622 by driving means 624, the latter of which may be positioned corporeally (intra- or extra-vascularly) or extracorporeally. As shown, the driving means 624 may comprise a motor 626 to which energy is supplied directly via an associated battery or an external power source, in a manner described in more detail herein. It is also contemplated that the impeller blade(s) 616 be driven electromagnetically through an internal or external electromagnetic drive. Preferably, a controller (not shown) is provided in association with this embodiment so that the pumping means 614 may be controlled to operate in a continuous and/or pulsatile fashion, as described herein.

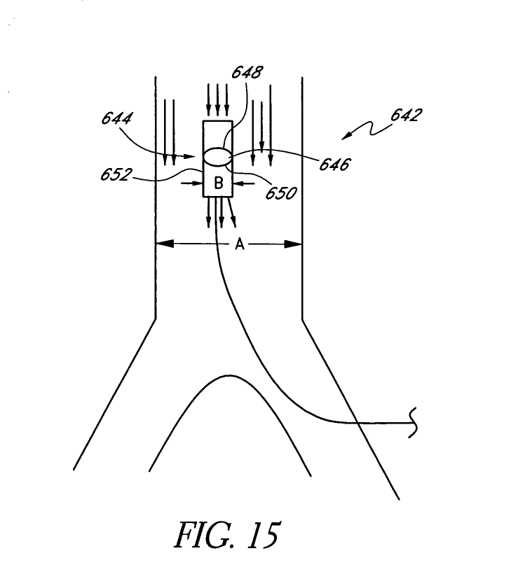

Variations of the intravascular embodiment of FIG. 14 are shown in FIGS. 15 and 16. In the embodiment of FIG. 15, an intravascular extracardiac system 642 comprises a pumping means 644, which may be one of several means described herein. The pumping means 644 may be driven in any suitable manner, including means sized and configured to be implantable and, if desired, implantable intravascularly, e.g., as discussed above. For a blood vessel (e.g., descending aorta) having a diameter “A”, the pumping means 644 preferably has a significantly smaller diameter “B”. The pumping means 644 may comprise a pump 646 having an inlet 648 and an outlet 650. The pumping means 644 also comprises a pump driven mechanically by a suitable drive arrangement in one embodiment. Although the vertical arrows in FIG. 15 illustrate that the pumping means 644 pumps blood in the same direction as the flow of blood in the vessel, the pumping means 644 could be reversed to pump blood in a direction generally opposite of the flow in the vessel.

In one embodiment, the pumping means 644 also includes a conduit 652 in which the pump 646 is housed. The conduit 652 may be relatively short, as shown, or may extend well within the designated blood vessel or even into an adjoining or remote blood vessel at the inlet end, at the outlet end, or at both the inlet and outlet. The intravascular extracardiac system 642 may further comprise an additional parallel-flow conduit, as discussed below in connection with the system of FIG. 16.

The intravascular extracardiac system 642 may further comprise inflow and/or outflow conduits or cannulae (not shown) fluidly connected to the pumping means 644, e.g., to the inlet and outlet of pump 646. Any suitable conduit or cannula can be employed. For example, a cannula defining a lumen with an inner size that increases distally could be coupled with an intravascular extracardiac system.

In another embodiment, an intravascular pumping means 644 may be positioned within one lumen of a multilumen catheter so that, for example, where the catheter is applied at the left femoral artery, a first lumen may extend into the aorta proximate the left subclavian and the pumping means may reside at any point within the first lumen, and the second lumen may extend much shorter just into the left femoral or left iliac. Such a system is described in greater detail in U.S. application Ser. No. 10/078,283, incorporated by reference herein above.

FIG. 16 shows a variation of the heart assist system of FIG. 15. In particular the intravascular system may further comprise an additional conduit 660 positioned preferably proximate the pumping means 644 to provide a defined flow path for blood flow axially parallel to the blood flowing through the pumping means 644. In the case of the pumping means 644 of FIG. 16, the means comprises a rotatable cable 662 having blood directing means 664 supported therein for directing blood axially along the cable. Other types of pumping means are also contemplated, if desired, for use with the additional conduit 660.

The intravascular extracardiac system described herein may be inserted into a patient's vasculature by any means known to those of ordinary skill, or by any obvious variant thereof. In one method of use, such a system is temporarily housed within a catheter that is inserted percutaneously, or by surgical cutdown, into a non-primary blood vessel and advanced through to a desired location. The catheter preferably is then withdrawn away from the system so as not to interfere with operation of the system, but to still permit the withdrawal of the system from the patient when desired. Further details of intravascular pumping systems may be found in U.S. patent application Ser. No. 10/686,040, filed Oct. 15, 2003, which is hereby incorporated by reference herein in its entirety.

C. Potential Enhancement of Systemic Arterial Blood Mixing

An advantage of the cardiac assist systems described above is the potential to enhance mixing of systemic arterial blood, particularly in the aorta. Such enhanced mixing ensures the delivery of blood with higher oxygen-carrying capacity to organs supplied by arterial side branches off of the aorta. A method of enhancing mixing using the cardiac assist systems described above preferably includes taking steps to assess certain parameters of the patient and then to determine the minimum output of the pump that, when combined with the heart output, ensures turbulent flow in the aorta, thereby enhancing blood mixing.

Blood flow in the aortic arch during normal cardiac output may be characterized as turbulent in the end systolic phase. Turbulence in a flow of fluid enhances the uniform distribution of particles within the fluid. It is believed that turbulence in the descending aorta enhances the homogeneity of blood cell distribution in the aorta. Laminar flow of viscous fluids leads to a higher concentration of particulate in the central portion of the flow. It is believed that, in low flow states such as that experienced during heart failure, there is reduced or inadequate mixing of blood cells leading to a lower concentration of nutrients at the branches of the aorta to peripheral organs and tissues. As a result, the blood flowing into branch arteries off of the aorta will likely have a lower hematocrit, especially that flowing into the renal arteries, the celiac trunk, the spinal arteries, and the superior and inferior mesenteric arteries, because these branches draw from the periphery of the aorta. The net effect of this phenomenon is that the blood flowing into these branch arteries has a lower oxygen-carrying capacity, because oxygen-carrying capacity is directly proportional to both hematocrit and the fractional O₂ saturation of hemoglobin. Under those circumstances, these organs may experience ischemia-related pathology.

The phenomenon of blood streaming in the aorta, and the resultant inadequate mixing of blood resulting in central lumenal concentration of blood cells, is believed to occur when the Reynolds number (N_{R}) for the blood flow in the aorta is below 2300. To help ensure that adequate mixing of blood will occur in the aorta to prevent blood cells from concentrating in the center of the lumen, a method of applying the cardiac assist systems described above to a patient may also include steps to adjust the output of the pump to attain turbulent flow within the descending aorta upstream of the organ branches; i.e., flow exhibiting a peak Reynolds number of at least 2300 within a complete cycle of systole and diastole. Because flow through a patient is pulsatile in nature, and not continuous, consideration is preferably given to how frequently the blood flow through the aorta has reached a certain desired velocity and, thus, a desired Reynolds number. The method contemplated herein, therefore, may also include the step of calculating the average Womersley number (N_{W}), which is a function of the frequency of the patient's heart beat. Preferably, a peak Reynolds number of at least 2300 is attained when the corresponding Womersley number for the same blood flow is approximately 6 or above.

More specifically, the method may comprise calculating the Reynolds number for the blood flow in the descending aorta by determining the blood vessel diameter and both the velocity and viscosity of the fluid flowing through the aorta. The Reynolds number may be calculated pursuant to the following equation:

$${N}_{R} = \frac{V \cdot d}{υ}$$

where: V=the velocity of the fluid; d=the diameter of the vessel; and υ=the viscosity of the fluid. The velocity of the blood flowing through the aorta is a function of the cross-sectional area of the aorta and the volume of flow therethrough, the latter of which is determined both by the patient's own cardiac output and by the output of the extra cardiac pump. Velocity may be calculated by the following equation:

$$V = \frac{Q}{π {r}^{2}}$$

where Q=the volume of blood flowing through the blood vessel, e.g., the aorta, per unit time; and r=the radius of the vessel. If the relationship between the pump output and the velocity is already known or independently determinable, the volume of blood flow Q may consist only of the patient's cardiac output, with the knowledge that that output will be supplemented by the subcardiac pump. If desired, however, the cardiac assist system can be implemented and applied to the patient first, before calculating Q, which would consist of the combination of cardiac output and the pump output.

The Womersley number may be calculated as follows:

$${N}_{W} = r \sqrt{2 π \frac{ω}{υ}}$$

where r is the radius of the vessel being assessed, ω is the frequency of the patient's heartbeat, and υ=the viscosity of the fluid. For a peak Reynolds number of at least 2300, a Womersley number of at least 6 is preferred, although lower values, such as 5, would also be acceptable.

By determining (i) the viscosity of the patient's blood, which is normally about 3.0 mm²/sec (kinematic viscosity), (ii) the cardiac output of the patient, which of course varies depending upon the level of CHF and activity, and (iii) the diameter of the patient's descending aorta, which varies from patient to patient but is about 21 mm for an average adult, one can determine the flow rate Q that would result in a velocity through the aorta necessary to attain a Reynolds number of at least 2300 at its peak during the patient's heart cycle. Based upon that determination of Q, one may adjust the output of the pump to attain the desired turbulent flow characteristic through the aorta, enhancing mixing of the blood therethrough.

One may use ultrasound (e.g., echocardiography or abdominal ultrasound) to measure the diameter of the aorta, which is relatively uniform in diameter from its root to the abdominal portion of the descending aorta. One may measure cardiac output using a thermodilution catheter or other techniques known to those of skill in the art. Finally, one may measure viscosity of the patient's blood by using known methods; for example, using a capillary viscosimeter. In many cases, the above methods will provide a basis to more finely tune the system to more optimally operate the system to the patient's benefit. Other methods may include steps to assess other patient parameters that enable a person of ordinary skill in the art to optimize the cardiac assist system to ensure adequate mixing within the vascular system of the patient.

Alternative methods that provide the benefits discussed herein include the steps of, prior to applying a shape change therapy, applying a blood supplementation system (such as one of the many examples described herein) to a patient, whereby the methods are designed to improve the ability to reduce the size and/or wall stress of the left ventricle, or both ventricles, thus reducing ventricular loading. Specifically, one example of such a method comprises the steps of providing a pump configured to pump blood at subcardiac rates, providing inflow and outflow conduits configured to fluidly communicate with non-primary blood vessels, fluidly coupling the inflow conduit to a non-primary blood vessel, fluidly coupling the outflow conduit to the same or different (primary or non-primary) blood vessel and operating the subcardiac pump in a manner, as described herein, to reduce the load on the heart, wherein the fluidly coupling steps may comprise anastomosis, percutaneous canalization, positioning the distal end of one or both conduits within the desired terminal blood vessel or any combination thereof. The method further comprises, after sufficient reduction in ventricular loading, applying a shape change therapy in the form of, for example, a cardiac reshaping device, such as those referred to herein, or others serving the same or similar function, for the purpose of further reducing the size of and/or wall stress on one or more ventricles and, thus, the heart, and/or for the purpose of maintaining the patient's heart at a size sufficient to enhance recovery of the patient's heart.

II. Method of Percutaneously Accessing High Flow Vessels

A variety of methods are discussed below for accessing a segment of the vasculature of a patient that is deeply buried, e.g., beneath organs and other soft tissues. The deeply buried segment of the vasculature to be accessed generally has a larger perimeter than segments of the vasculature that are close to the skin, e.g., segments that can be accessed by conventional percutaneous techniques. These vessel segments are at least in this sense relatively large. The deeply buried vessel segment generally has a relatively high flow capacity due to its size. These methods can involve accessing such vessels by way of peripheral vessels and methods of directly accessing high flow vessels from a percutaneous site above the high flow vessel.

A. Accessing Large Perimeter Vessel Segments From Other Vessel Segments

In a class of methods, a clinician is able to access a deeply buried, relatively large (e.g., high volume flow) portion of the vasculature for, among other applications, supplementing blood flow in a manner described herein or otherwise. To avoid the need for surgical cut-down, but without sacrificing the use of a high volume flow catheter, one method of the present invention comprises accessing the deeply buried target vasculature site with a catheter directed through a proximate first percutaneous site by way of a second percutaneous site. In that regard, one method comprises the step of puncturing the patient's skin and vasculature with a needle at the second percutaneous site remote from the target location and then inserting a guide wire through the needle and into the vasculature at the second percutaneous site. The needle is then removed from the vasculature and the guide wire is advanced through the vasculature to the target location. To ensure that the guide wire reaches the target location, and to follow the progress of the guide wire along the way, the guide wire may be tracked with the aid of visualization apparatus. For example, the visualization apparatus may comprise a fiber-optic camera, ultrasound apparatus or fluoroscopy apparatus. Those of skill in the art will appreciate that other visualization apparatus could be used in addition to, or instead of, the examples listed above.

The needle produces relatively small openings in the skin and in the vasculature at the second percutaneous site. Therefore, it is often advantageous to widen these openings using one or more dilators. To do so, at least one dilator may be advanced over the guide wire and inserted into the vasculature at the second percutaneous site. The dilator widens the openings in the vasculature and the skin at the second percutaneous site. The openings may be widened a little bit at a time by using successively larger dilators. This process is known as step dilation, and is well known by those of skill in the art.

When the openings in the vasculature and the skin at the second percutaneous site are sufficiently wide, a tunneling device is advanced along the guide wire and into the vasculature. For example, the tunneling device may comprise a tunneling catheter having a distal tip. As discussed further below, the distal tip can have a structure configured to pierce a portion of the vasculature of the patient, e.g., a vessel wall. As discussed further below, the distal tip or vasculature piercing structure may be covered, e.g., by being at least temporarily retracted within a portion of the catheter. In some embodiments, a cover may be provided that is retractable to expose the distal tip or vasculature piercing structure. In one method, the tunneling device is advanced along the guide wire through the vasculature to the target location. For this step, visualization apparatus may again be used.

Once the tunneling device distal tip reaches the target location, a cover is removed, if one has been provided. In some embodiments, the cover can comprise a sheath that can be moved relative to the distal tip or relative to the vasculature piercing structure to expose or to release the tip or structure. The sheath extends over at least a portion of the outer surface of the distal tip or vasculature piercing structure in some embodiments. The sheath is a structure that prevents harmful interaction between the vasculature and the distal tip or vasculature piercing structure prior to intended piercing of a vessel wall at the target location. In other embodiments, the distal tip or vasculature piercing structure can be configured to be extended distally to expose a portion of the tunneling device, e.g., the distal tip or vasculature piercing structure, adjacent to the target location. The distal tip of the tunneling device is then manipulated to pierce the vasculature wall at the target location. The tunneling device is advanced through the vasculature wall and through the patient's tissue to the first percutaneous site. During the tunneling process, any suitable technique may be used to prevent excess bleeding at the target location or in the intervening tissue. In order to avoid sensitive bodily structures, a visualization apparatus may again be used.

The tunneling device provides a path from the first percutaneous site to the target location. A guidewire may also be used to telegraph the pathway. After the pathway has been created, the tunneling device may be removed from the pathway. If the tunneling device has been removed from the pathway, then a sheath can be advanced along the pathway to the vasculature proximate the target location. However, if the tunneling device has been left in place, then a sheath is advanced over the tunneling device to the vasculature proximate the target location. A visualization apparatus may be employed for these steps.

A distal end of the sheath preferably includes an apparatus that is configured to capture the vasculature. For example, at the distal end of the sheath, opposing side walls may include first and second arcuate cutout portions. The cutouts are adapted to receive the vessel, such that the vessel runs substantially perpendicular to a longitudinal axis of the sheath, and the cutouts at least partially surround the vessel. With the aid of the capturing apparatus, the vasculature is captured. Visualization apparatus may be employed to complete this step. If the tunneling device is still resident within the sheath, it can be removed at this point. A cannula can be passed to the target location through the sheath or tunneling device or over the guide wire.

The tunneling device, sheath, and/or the guide wire may thus be used to guide a cannula through the first percutaneous site, through the patient's tissue, and to or into the vasculature at the target location. The method described above may be used to access any of a wide variety of target locations. For example, the target location may be the abdominal aorta or the vena cava. By using the method described herein, these targets may be accessed through any of a wide variety of second percutaneous sites. For example, the second percutaneous site may be located at the axillary artery, the iliac artery of vein, the femoral artery or vein, the subclavian artery or vein, the common carotid artery, the brachiocephalic artery, the great saphenous vein, the internal or external jugular vein, or the basilic vein.

B. Accessing Large Perimeter Vessel Segments from Above a Target Location

If desired, an alternate application of the present inventive method comprises puncturing a patient's skin with a needle at the first percutaneous site; inserting a tunneling device through the patient's skin at the percutaneous site; advancing the tunneling device through the patient's tissue beneath the percutaneous insertion site, with the aid of a visualization apparatus, so as to avoid sensitive bodily structures, to the vasculature, thereby creating a pathway from the percutaneous insertion site to the vasculature proximate the target location; removing the tunneling device from the pathway; advancing a sheath, with the further aid of the visualization apparatus, along the pathway to the vasculature proximate the target location, an end of the sheath including apparatus configured to capture the vasculature; capturing the vasculature with the capturing apparatus and with the further aid of the visualization apparatus; advancing a guide wire through the sheath to the vasculature, with the further aid of the visualization apparatus; advancing a needle along the guide wire to the vasculature, with the further aid of the visualization apparatus; piercing a wall of the vasculature with the needle, and with the further aid of the visualization apparatus, to produce a vascular opening; advancing the guide wire through the vascular opening, with the further aid of the visualization apparatus, and into the vasculature at the target location; removing the needle from the vascular opening; advancing a dilator along the guide wire, with the further aid of the visualization apparatus, and through the vascular opening to widen the opening; and advancing a cannula along the guide wire, with the further aid of the visualization apparatus, through the vascular opening, and into the vasculature at the target location.

As discussed above, one variation involves accessing a large or relatively large perimeter vessel (e.g., a relatively high flow vessel) from above the target location. This technique is sometimes referred to herein as accessing the target location directly. Here, “directly” and “direct access” are broad terms and they include access a vessel or a vessel segment at a target location without the need previously to insert a guide wire, tunneling device, or other structure into the vasculature at another location or through another vessel segment. These and similar terms also include techniques that create a pathway primarily through non-vascular tissues, as discussed below.

Direct access methods can be facilitated by securing the vasculature at the target location. In one technique, the sheath with cutout portions adapted to receive a vessel, which is discussed above, is used to secure the vasculature at a target location. Once the vasculature has been secured, a guide wire is advanced through the sheath to the vasculature in one technique. A needle is then advanced along the guide wire to the vasculature. The needle pierces a wall of the vasculature. Visualization apparatus may be used to complete these steps. The needle produces a vascular opening, through which the guide wire is advanced into the vasculature at the target location. When the needle is removed from the vascular opening, the vascular opening tends to close up and assume the size of the guide wire. Therefore, in order to widen the vascular opening, a dilator may be advanced along the guide wire and through the vascular opening. A series of progressively larger dilators may be advanced through the vascular opening until it achieves the desired size. Once the vascular opening is large enough, a cannula is advanced along the guide wire, through the vascular opening, and into the vasculature at the target location. At each of the above steps, a visualization apparatus may be employed.

In the above methods, the tunneling device may comprise a removable core with a tunneling tip. With such a tunneling device, once the device reaches the vasculature at the target location, the capturing apparatus is extended from the tunneling device to capture the vasculature. The core is then removed from the tunneling device, leaving behind a sheath. The remaining steps may proceed as described above.

In performing the above methods, one of a number of possible tunneling devices may be used, with optional features that may enhance operation. The tunneling device preferably includes a distal end that is configured to penetrate the vasculature and tissue between the target vasculature site and the first percutaneous site. For example, the distal end may include a simple dissection tip, a trocar-type tip, a hollow catheter with an extendable cutter, a blunt dissection tip, a laser tip, an RF tip, an electrosurgical tip, or an ultrasound tip. If the distal tip of the tunneling device is sharp, then in order to prevent damage to the vasculature, a removable cover may shield the distal tip as the tunneling device travels through the vasculature.

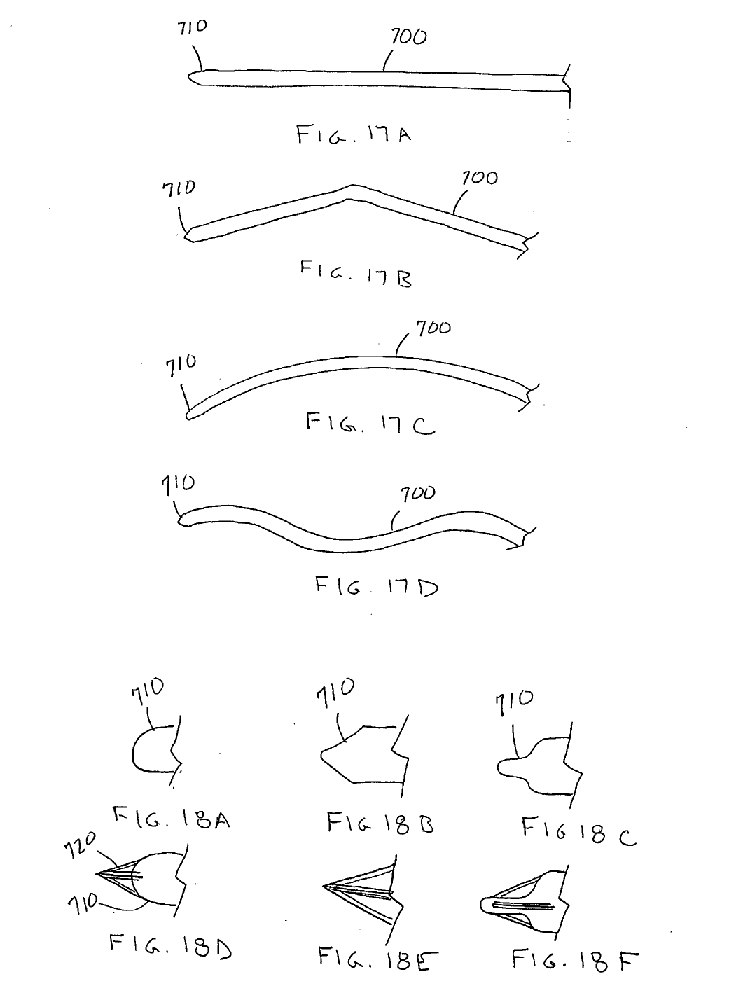

Referring to FIG. 17A through FIG. 17D, one embodiment of a tunneling device 700 comprises a distal tip 710 and may be formed with one of several configurations, including straight, angled, curved or free form, respectively. The material for each tunneling device, or at least a distal portion thereof, may be made of shape memory alloys, if desired, or more rigid material, depending upon the circumstances. Referring to FIGS. 18A-C, the distal tip 710 may have one or several configurations, as shown. If desired, a trocar-style tip may be employed having one or more blades 720, as shown in FIGS. 18D-F, where the blades may be straight or curved. If desired, a cutting tip may be retractable and extendable. Referring to FIGS. 19A-B, the tunneling device 700 may comprises a retractable blade 730 that may be extended remotely as needed. This feature permits the blade 730 to remain retracted during travel, but extend for use in cutting through tissue as desired. Various cutting edges may be employed, as referring to above. An alternative embodiment is shown in FIG. 20, in which the tunneling device 700 comprises an extendable rod 740, or other suitable carrying device, for supporting a cutting device 750 thereon. Other variations are contemplated for providing a tunneling device that is capable of selectively cutting through tissue in a desired direction.

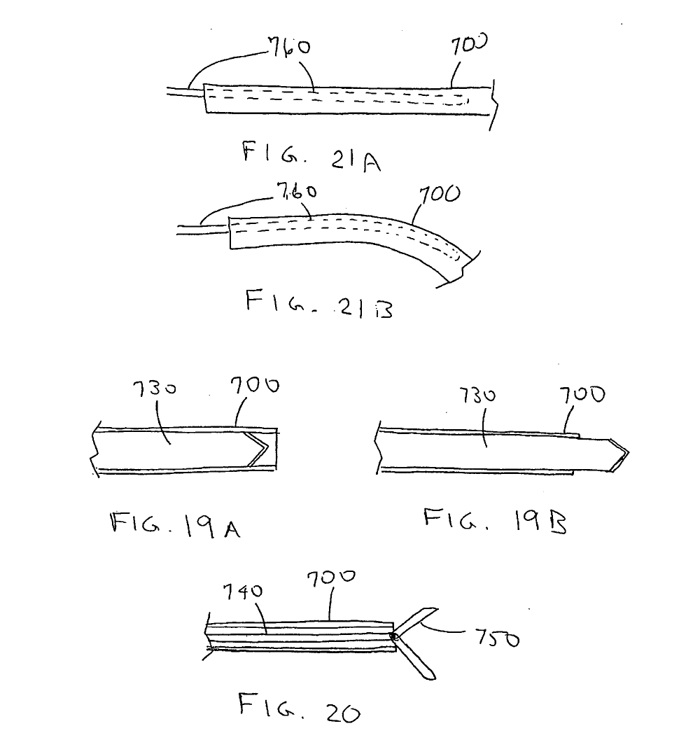

If desired, the tunneling device 700 may be configured to be steerable by the clinician. Referring to FIGS. 21A and 21B, the tunneling device 700 may comprises a steerable component 760, such as a control wire, extended at least partially therethrough. With such a feature, the tunneling device 700 can be remotely operated so as to steer the tunneling device 700 in a particular direction, helpful but not necessary in exiting the target vasculature site for penetration through the intervening tissue separating the target vasculature site from the first penetration site.

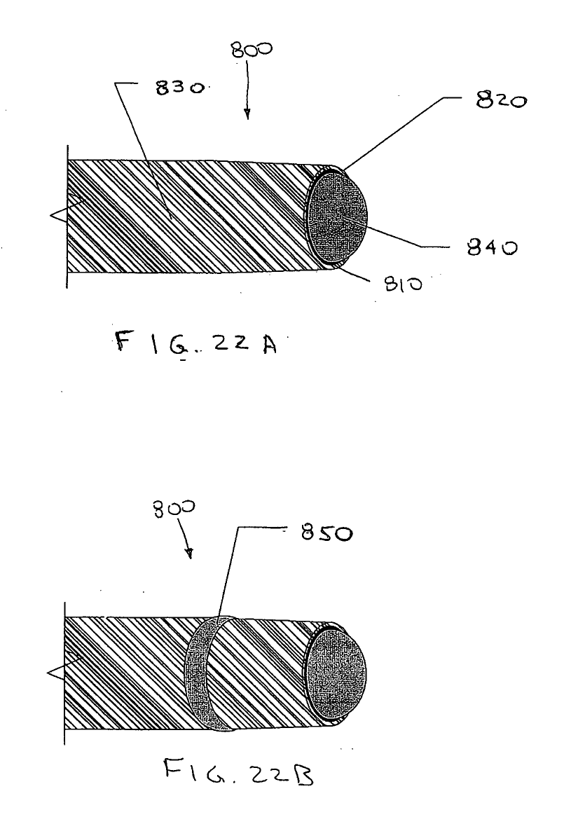

Referring to FIGS. 22A-B, an alternative tunneling device 800 comprises a tube 810 with a high temperature insulating layer thereon. If desired, a lubricious coating 830 may be applied thereto. The tube 810 supports at a distal end 820 an electrode 840 of one of many possible configurations to burn away tissue, to pierce the vasculature, and to cut through tissue while tunneling. The electrode 840 may be retractable. The tunneling device 800 may also be steerable. Referring to FIG. 22B, the tunneling device 800 may further comprises a cauterizing portion 850 that may be used to close open tissue following the cutting step. The cauterizing portion 850 may be configured in one of a number of different configurations.

The present method may be used to reach a target location that is not a treatment site at which the vascular procedure is desired to be performed. In such a case, the target location may be selected because it is a location at which a relatively large vascular instrument may be inserted and then advanced intravascularly to a treatment location. For example, the vascular instrument may be a cannula, and the target location may be in the iliac artery, while the treatment location is in the aortic arch. The cannula may thus be inserted into the iliac artery through the first percutaneous site, and then advanced to the aortic arch to perform the desired treatment. Additional examples of treatment locations include the abdominal aorta, the axillary artery or vein, and the inferior or superior vena cava.

The above steps illustrate some examples of the present inventive methods and present a description of the best mode contemplated for carrying out the present methods for minimally invasive vascular access, and of the manner and process of performing them, in such full, clear, concise, and exact terms as to enable any person skilled in the art to which they pertain to practice these methods. These methods are, however, susceptible to modifications and alternate apparatus from that discussed above that are fully equivalent. Consequently, these methods are not limited to the particular embodiments disclosed. On the contrary, these methods cover all modifications and alternate constructions coming within the spirit and scope of the methods as generally expressed by the following claims, which particularly point out and distinctly claim the subject matter of the methods.

What is claimed is:

1. A method for minimally invasive access to a target location in a patient's vasculature, the target location being buried deep beneath the patient's skin such that a relatively large amount of bodily tissue and/or organs lie between a first percutaneous site and the target location, the target location being accessible from a second percutaneous site where the vasculature is located relatively close to the skin, the vasculature at the target location including a vessel segment with a first perimeter that is larger than a second perimeter of a second vessel segment located near the second percutaneous site, the method comprising the steps of:
puncturing a patient's skin and vasculature with a needle at the second percutaneous site;
inserting a guide wire through the needle and into the vasculature at the second percutaneous site;
removing the needle from the vasculature;
advancing the guide wire through the vasculature, with the aid of a visualization apparatus, to the target location;
advancing a dilator over the guide wire and inserting the dilator into the vasculature at the second percutaneous site, thereby widening an opening in the vasculature at the second percutaneous site;
advancing a tunneling device having a cover through the vasculature, with the further aid of the visualization apparatus, along the guide wire from the second percutaneous site to the target location, the tunneling device being configured to be steerable and having a distal point capable of penetrating the vasculature and tissue between the vasculature and the skin, the cover protecting the vasculature as the device travels through the vasculature;
piercing the vasculature wall with the tunneling device at the target location and advancing the tunneling device through the vasculature wall and through the patient's tissue, with the further aid of the visualization apparatus, avoiding sensitive bodily structures, to the first percutaneous site; and
inserting a conduit through the first percutaneous site, through the patient's tissue, and into the vasculature at the target location.

2. The method of claim 1, wherein the second percutaneous site is proximate an artery.

3. The method of claim 2, wherein the artery is the axillary artery, the femoral artery, the subclavian artery, the common carotid artery, or the brachiocephalic artery.

4. The method of claim 1, wherein the second percutaneous site is proximate a vein.

5. The method of claim 4, wherein the vein is the femoral vein, the great saphenous vein, the internal or external jugular vein, the subclavian vein or the basilic vein.

6. The method of claim 1, wherein the target location is in an artery.

7. The method of claim 6, wherein the artery is the common femoral artery, the common iliac artery, the abdominal aorta, the axillary artery, the subclavian artery, or the brachiocephalic artery.

8. The method of claim 1, wherein the target location is in a vein.

9. The method of claim 8, wherein the vein is the common femoral vein, the common iliac vein, the inferior vena cava, the superior vena cava, the axillary vein, the subclavian vein, the external or the internal jugular vein, or the basilic vein.

10. The method of claim 1, wherein the visualization apparatus comprises one or more of a fiber-optic camera, ultrasound apparatus or fluoroscopy apparatus.

11. The method of claim 1, further comprising the step of advancing the conduit from the target location to the aortic arch, the abdominal aorta, the axillary artery, the inferior or superior vena cava, the axillary vein, or the renal artery.

12. The method of claim 1, wherein the tunneling device comprises a tunneling catheter.

13. The method of claim 12, wherein the tunneling device further comprises a dissection tip, a trocar tip, a hollow catheter with an external cutter, a blunt dissection tip, a laser tip, an RF tip, an electrosurgical tip, or an ultrasound tip.

14. A method for minimally invasive access to a target location in a patient's vasculature, the target location being buried deep beneath the patient's skin such that a relatively large amount of bodily tissue and/or organs lie between a first percutaneous site and the target location, the target location being relatively easy to access from a second percutaneous site where the vasculature is located relatively close to the skin, the vasculature at the target location including a vessel segment with a first perimeter that is larger than a second perimeter of a second vessel segment located near the second percutaneous site, the method comprising the steps of:
advancing a tunneling device into the vasculature at the second percutaneous site;
advancing the tunneling device through the vasculature from the second percutaneous site to the target location; and
advancing the tunneling device through the vasculature at the target location, through the patient's tissue, and through the patient's skin at the first percutaneous site.

15. The method of claim 14, further comprising the step of inserting a conduit through the first percutaneous site, through the patient's tissue, and into the vasculature at the target location.

16. A method for minimally invasive access to a target location in a patient's vasculature that includes a vessel segment with a relatively large perimeter, the target location being buried deep beneath the patient's skin, such that a relatively large amount of bodily tissue and/or organs lie between a percutaneous site and the target location, the method comprising the steps of:
puncturing a patient's skin with a needle at the percutaneous site;
inserting a tunneling device through the patient's skin at the percutaneous site;
advancing the tunneling device through the patient's tissue beneath the percutaneous insertion site, with the aid of a visualization apparatus, so as to avoid sensitive bodily structures, to the vasculature, thereby creating a pathway from the percutaneous insertion site to the vasculature proximate the target location;
removing the tunneling device from the pathway;
advancing a sheath, with the further aid of the visualization apparatus, along the pathway to the vasculature proximate the target location, an end of the sheath including apparatus configured to capture the vasculature;
capturing the vasculature with the capturing apparatus and with the further aid of the visualization apparatus;
piercing a wall of the vasculature with a hollow structure, and with the further aid of the visualization apparatus, to produce a vascular opening;
removing the hollow structure from the vascular opening;
advancing a dilator along the guide wire, with the further aid of the visualization apparatus, and through the vascular opening to widen the opening; and
advancing a conduit along the guide wire, with the further aid of the visualization apparatus, through the vascular opening, and into the vasculature at the target location.

17. The method of claim 16, further comprising:
advancing a guide wire through the sheath to the vasculature, with the further aid of the visualization apparatus;
advancing the hollow structure along the guide wire to the vasculature, with the further aid of the visualization apparatus; and
advancing the guide wire through the vascular opening, with the further aid of the visualization apparatus, and into the vasculature at the target location.

18. The method of claim 16, wherein the target location is in an artery.

19. The method of claim 18, wherein the artery is the common femoral artery, the common iliac artery, the abdominal aorta, the axillary artery, the subclavian artery, or the brachiocephalic artery.

20. The method of claim 16, wherein the target location is in a vein.

21. The method of claim 20, wherein the vein is the common femoral vein, the common iliac vein, the inferior vena cava, the superior vena cava, the axillary vein, the subclavian vein, the external or the internal jugular vein, or the basilic vein.

22. The method of claim 16, wherein the visualization apparatus comprises one or more of a fiber-optic camera, ultrasound apparatus or fluoroscopy apparatus.

23. The method of claim 16, further comprising the step of advancing the conduit from the target location to the aortic arch, the abdominal aorta, the axillary artery, the inferior or superior vena cava, the axillary vein, or the renal artery.

24. The method of claim 16, wherein the tunneling device comprises a simple dissection tip, a trocar-type tip, a hollow catheter with an extendable cutter, a blunt dissection tip or a laser tip.

25. The method of claim 16, wherein the tunneling device is straight, angled, curved or anatomically shaped.

26. A method for minimally invasive access to a target location in a patient's vasculature that includes a vessel segment with a relatively large perimeter, the target location being buried deep beneath the patient's skin, such that a relatively large amount of bodily tissue and/or organs lie between a percutaneous site and the target location, the method comprising the steps of:
puncturing a patient's skin with a needle at the percutaneous site;
inserting a tunneling device through the patient's skin at the percutaneous site, the tunneling device comprising a removable core with a tunneling tip;
advancing the tunneling device through the patient's tissue beneath the percutaneous insertion site, with the aid of a visualization apparatus, so as to avoid sensitive bodily structures, to the vasculature, thereby creating a pathway from the percutaneous insertion site to the vasculature proximate the target location;
extending capturing apparatus from the tunneling device and, with the further aid of the visualization apparatus, capturing the vasculature with the capturing apparatus;
removing the core from the tunneling device;
advancing a hollow structure through the tunneling device to the vasculature, with the further aid of the visualization apparatus;
piercing a wall of the vasculature with the hollow structure, and with the further aid of the visualization apparatus, to produce a vascular opening;
removing the hollow structure from the vascular opening;
advancing a dilator through the patient's tissue, with the further aid of the visualization apparatus, and through the vascular opening to widen the opening;
advancing a conduit through the patient's tissue, with the further aid of the visualization apparatus, through the vascular opening, and into the vasculature at the target location.

27. The method of claim 26, further comprising:
advancing a guide wire through the tunneling device to the vasculature, with the further aid of the visualization apparatus;
advancing the hollow structure along the guide wire to the vasculature, with the further aid of the visualization apparatus; and
advancing the guide wire through the vascular opening, with the further aid of the visualization apparatus, and into the vasculature at the target location;

28. A method for minimally invasive access to a target location in a patient's vasculature that includes a vessel segment with a relatively large perimeter, the target location being buried deep beneath the patient's skin, such that a relatively large amount of bodily tissue and/or organs lie between a percutaneous site and the target location, the method comprising the steps of:
inserting a tunneling device through a patient's skin at the percutaneous site;
advancing the tunneling device through the patient's tissue beneath the percutaneous site to the vasculature proximate the target location, thereby creating a pathway from the percutaneous site to the vasculature proximate the target location;
piercing a wall of the vasculature to create a vascular opening; and
advancing a conduit along the pathway, through the vascular opening, and into the vasculature at the target location.

29. A tunneling device for permitting a clinician to reach a target location of a patient's vasculature that is deeper than subcutaneous from a percutaneous site that is remote from the target portion, the tunneling device comprising an elongate portion configured to pass through the patient's vasculature, the elongate portion having a distal end configured to cut through the vasculature and tissue.

30. The tunneling device of claim 29, wherein the distal end comprises a cutting edge.

31. The tunneling device of claim 30, wherein the cutting edge is retractable.

32. The tunneling device of claim 29, wherein the tunneling device is remotely steerable.
